(19) United States
(12) Patent Application Publication
(10) Pub. No.: US 2007/0048803 A1
(43) Pub. Date: Mar. 1, 2007

(54) Immune profiling of lymph nodes associated with cancer

(76) Inventors: Peter Poon-Hang Lee, Menlo Park, CA (US); Holbrook Edwin Kohrt, Santa Clara, CA (US); Susan P. Holmes, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/509,976

(22) Filed: Aug. 25, 2006

Related U.S. Application Data
(60) Provisional application No. 60/711,993, filed on Aug. 25, 2005.

(51) Int. Cl. G01N 33/567

(52) U.S. Cl. 435/7.2

(57) ABSTRACT

Cancers are phenotyped by analysis of the presence of immune cells, particularly T cells and dendritic cells, present in regional lymph nodes. When non-sentinel regional lymph nodes of a cancer patient are compared to normal lymph nodes, it is found that increased number of dendritic cells and decreased numbers of T cells, particularly T helper cells, correlate with a positive prognosis and disease-free survival following conventional chemotherapy.

Cancers of different types are typically classified, or staged, according to the spread of the primary tumor. For example, the TNM staging system, (Tumor, lymph Nodes, Metastasis) is widely used. Using the TNM staging system, information about the tumor, lymph nodes, and metastasis is combined to assign a stage. Metastasis to the lymph nodes is clearly recognized as an important component in assessing the stage of many cancers.

For example, if a breast cancer was suspected of being invasive, traditionally an axillary lymph node dissection was performed to determine if the cancer had spread to the lymph nodes. During such a dissection, the bulk of the lymph node tissue that drains from the breast was removed and histologically examined. Sentinel lymph node (SLN) biopsy provided an alternative technique, which has also been widely applied to other types of cancer. The lymph ducts of the breast usually drain to one lymph node first, before draining through the rest of the lymph nodes underneath the arm. The first lymph node is called the sentinel lymph node. Lymph node mapping identifies the sentinel using a dye tracer, usually a colored or radioactive dye, then removes only the sentinel node for analysis. With the growing practice of SLN biopsy, new methods of lymph node analysis are being developed. SLN evaluation by multiple hematoxylin and eosin stained sections (HES), immunohistochemistry (IHC), and most recently, reverse-transcription polymerase chain reaction (RT-PCR) for breast cancer-associated gene expression has increased metastasis detection by up to 42%. Despite these technical advances, the prognostic significance of isolated tumor cells and RT-PCR positive nodes remains inconclusive and highly debated.

Concurrent advances in pathological analysis of primary tumors have found infiltrating immune cells of prognostic significance (see, for example Coventry and Morton (2003) Br J Cancer 89:533-538). Detailed histological analyses identified tumor-infiltrating T lymphocytes and dendritic cells, with diminished dendritic cell infiltration directly correlated with increased nodal metastasis and poor disease-free and overall survival (Georgiannos et al. (2003) Surgery 134:827-834; Liyanage et al. (2002) J Immunol 169:2756-2761; Iwamoto et al. (2003) Int J Cancer 104:92-97; and Lespagnard et al. (1999) Int J Cancer 84:309-314). Decreased circulating T lymphocyte populations have also been shown to correlate with poor overall survival (Blake-Mortimer et al. (2004) Breast J 10: 195-199).

Substantial evidence now exists showing impairment of the systemic and local immune response during cancer progression. However, it is often overlooked that local tumor-draining nodes are the immunologically active sites where such immune responses, including tumor antigen presentation and lymphocyte activation, should develop. Alterations of the immune response might be associated with lymph node invasion by tumor, and may precede microscopic metastasis detection. The present invention addresses these issues.

### SUMMARY OF THE INVENTION

Methods are provided for the clinical phenotyping of cancers by profiling the immune cells of regional lymph nodes associated with the cancer. The immune profile analysis may specifically exclude analysis of the sentinel node. The lymph node immune profile, which may include quantitation of T lymphocyte and dendritic cell populations, reflects alterations in immune cells present in the region of the tumor, and is shown herein to be useful in determination of patient prognosis. The phenotyping methods provided herein allow for improved patient care by determining patient prognosis and allowing for appropriate intervention.

In the methods of the invention, lymph node samples from nodes associated with a cancer are differentially analyzed for the presence of immune cells, which immune cells include dendritic and T cell populations, where the distribution of cells is diagnostic of the invasiveness of the cancer. A utility of particular interest is the evaluation of axillary nodes for breast cancer patients, where a prediction of disease-free survival can be made.

In one embodiment of the invention, a regional lymph node sample from a cancer patient, which may specifically include or exclude the sentinel lymph node, is stained with reagents specific for CD4; and for CD1a, and may optionally include a reagent specific for CD8. Additional markers of interest include CD25, and FoxP3. The analysis of staining patterns in the lymph node cells provides the relative distribution of immune cells, which distribution predicts the cancer stage and prognosis. In some embodiments, the patient sample is compared to a control, or a standard test value. In other embodiments, the patient sample is compared to a pre-cancer sample, or to one or more time points through the course of the disease.

In breast cancer, it is shown that sentinel and non-sentinel axillary node T cells are decreased compared to control nodes. Dendritic cells are decreased in sentinel, but increased in non-sentinel axillary nodes. Axillary node, but not sentinel node, CD4⁺ T cell and dendritic cell populations are highly correlated with disease-free survival, independent of axillary metastasis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1N. Lymph Node Profile of Sentinel and Axillary Lymph Nodes. Mean and standard error of CD4 and CD 8 T cell, CD1a dendritic cell populations as percent of lymph node and CD4:CD8 cell ratio are shown for sentinel (n=29)(SLN), axillary (n=29)(ALN), and control lymph nodes (n=10)(Panel A); tumor-involved ALNs (n=9), tumor-free ALNs (n=7) from patients with a positive ALN dissection (ALND), tumor-free ALNs from patients with a negative ALND (n=13), and controls (n=10)(Panel E); SLNs and ALNs stratified by disease recurrence during 60 months of follow-up (11 of 29 with recurrent disease)(Panel I); tumor-involved ALNs stratified by disease recurrence (n=9)(Panel L); tumor-free ALNs from patients with a positive ALND stratified by disease recurrence (n=7)(Panel M); and tumor-free ALNs from patients with a negative ALND stratified by disease recurrence (n=13)(Panel N). Representative 200× images of lymphocyte population (brown staining) and infiltrating tumor (purple staining) by immunohistochemistry, including CD8 T cells in SLNs (Panel B), ALNs (Panel C), and controls (Panel D); CD4 T cells in tumor-involved ALNs (Panel F), tumor-free ALNs from patients with a positive ALND (Panel G), and tumor-free ALNs from patients with a negative ALND (Panel H); and CD1a dendritic cells in ALNs from patients disease-free (Panel J) versus patients who developed recurrence (Panel K).

FIGS. 2A-2C. Disease-free Survival Analysis of Women with Breast Cancer According to Tumor and Immune profile Characteristics, Learning Set ALN Series 1. Kaplan-Meier curves are shown for median disease-free survival (DFS) applied to the learning set, n=29, according to percent of sentinel node (SLN) occupied by infiltrating tumor (determined by immunohistochemistry), and stratified by tumor stage (Panel A); DFS according to size of CD4 T cell and CD1a dendritic cell populations within learning set axillary lymph node (ALN) series 1 (first, arbitrarily selected ALN per subject)(Panel B); and DFS stratified by both percent of SLN infiltrated by tumor and tumor stage, and second, by both axillary node CD4 T cell population and by tumor stage (Panel C). Panel C includes comparison of survival by all subgroups and a separate comparison of stratified T2 alone, *. Thresholds for percent tumor infiltration within SLN, ALN CD4 T cell and ALN CD1a dendritic cell populations were determined by receiver-operating-characteristic curves as applied to the learning set (SLN and ALN series 1). Median duration of DFS are indicated; -- median DFS greater than follow-up period, 60 months; TI, tumor infiltration. 11 of 29 subjects with recurrent disease. Adjusted P values were determined by the permuted log-rank statistic for comparison of disease-free survival between groups.

FIGS. 3A-3C. Disease-free Survival Analysis of Women with Breast Cancer According to Immune profile Characteristics, Learning Set ALN Series 2 and Test Set. Kaplan-Meier curves are shown for median disease-free survival (DFS) applied to the learning set ALN series 2 (n=27) and test set (n=48) according to size of CD4 T cell and CD1a dendritic cell populations within learning set axillary node (ALN) series 2 (second, randomly selected ALN per subject)(Panel A); DFS stratified by size of CD4 T cell and CD1a dendritic cell populations within test set ALNs (Panel B); and DFS applied to the learning set ALN series 1 (n=29) series 2 (n=27) and test set (n=48) according to size of CD4 T cell and CD1a dendritic cell populations within ALN (Panel C). Thresholds for ALN CD4 T cell and ALN CD1a dendritic cell populations were determined by receiver-operating-characteristic curves as applied to the learning set (ALN series 1). Median duration of DFS are indicated; -- median DFS greater than follow-up period, 60 months. 11 of 29 subjects with recurrent disease are in learning set ALN series 1, and 11 of 27 subjects with recurrent disease in learning set ALN series 2. 22 of 48 subjects with recurrent disease in test set. Adjusted P values were determined by the permuted log-rank statistic for comparison of disease-free survival between groups.

FIGS. 4A-4C. Disease-free Survival Analysis of Women with Breast Cancer According to Tumor Stage, T1 and T2, and Immune profile Characteristics, Learning Set ALN Series 1 and 2 and Test Set. Kaplan-Meier curves are shown for median disease-free survival (DFS) applied to the learning set ALN series 1 (n=29) series 2 (n=27) and test set (n=48) according to tumor stage, number of tumor-involved ALNs (nonsentinel) among subjects with T1 tumors, and number of negative SLNs among subjects with T2 tumors (Panel A); DFS stratifies by size of CD4 T cell and CD1a dendritic cell populations within ALNs among subjects with T1 tumors (Panel B); and DFS stratifies by size of CD4 T cell and CD1a dendritic cell populations within ALNs among subjects with T2 tumors (Panel C). Thresholds for ALN CD4 T cell and ALN CD1a dendritic cell populations were determined by receiver-operating-characteristic curves as applied to the learning set (ALN series 1). Median duration of DFS are indicated; -- median DFS greater than follow-up period, 60 months. 44 of 104 ALNs from subjects with disease recurrence. 20 of 56 ALNs from subjects with T1 tumors with recurrent disease. 19 of 43 ALNs from subjects with T2 tumors with disease recurrence. Adjusted P values were determined by the permuted log-rank statistic for comparison of disease-free survival between groups.

FIG. 5. Axillary Lymph Node Status, Learning Set Series 1. Axillary lymph node (ALN) dissection was positive in 16 of 29 subjects. Tumor-involvement was determined for a single ALN (nonsentinel) per subject (learning set ALN series 1)(n=29). Nine ALNs contained tumor infiltration. Of 20 tumor-free ALNs, seven were selected from patients with a positive ALN dissection and thirteen from patients with a negative ALN dissection.

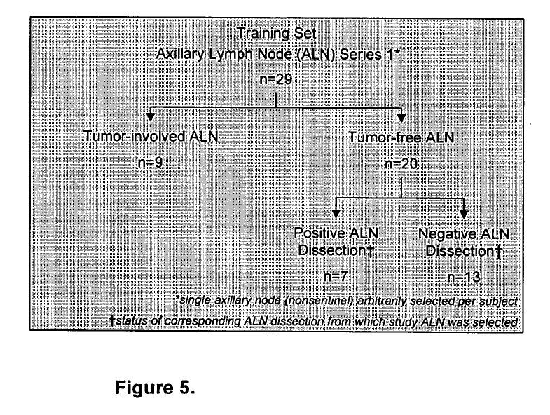

FIG. 6. Axillary Lymph Node Status, Learning Set Series 2. Axillary lymph node (ALN) dissection was positive in 16 of 29 subjects. Tumor-involvement was determined for a single ALN (nonsentinel) per subject (learning set ALN series 2)(n=27). Seven ALNs contained tumor infiltration. Of 20 tumor-free ALNs, eight were selected from patients with a positive ALN dissection and 12 from patients with a negative ALN dissection.

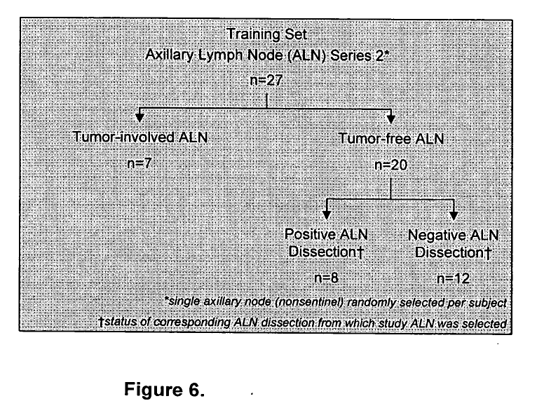

FIG. 7. Axillary Lymph Node Status, Test Set. Axillary lymph node (ALN) dissection was positive in 31 of 48 subjects. Tumor-involvement was determined for a single ALN (nonsentinel) per subject (test set)(n=48). Seventeen ALNs contained tumor infiltration. Of 31 tumor-free ALNs, 14 were selected from patients with a positive ALN dissection and 17 from patients with a negative ALN dissection.

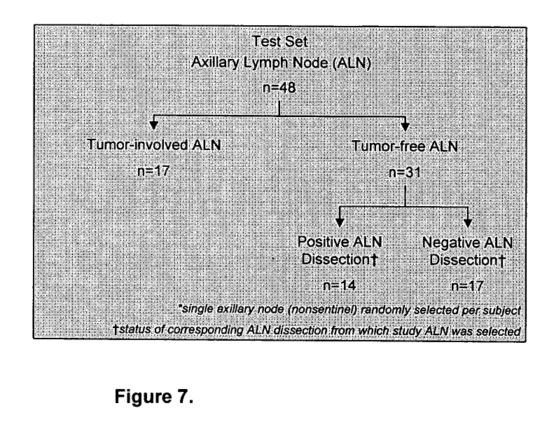

FIG. 8. Predictive Strength of Patient and Immune Profile Characteristics, Learning Set. Panel A shows the receiver-operating-characteristic (ROC) curve calculating the sensitivity and specificity of lymph node CD4 T cell, CD1a dendritic cell and the ratio of CD4:CD8 T cell populations in detecting nodal metastases from the learning set (sentinel lymph node, SLN, n=29, and axillary lymph node, ALN, series 1, n=29). Panel B shows the ROC curve calculating the sensitivity and specificity of first-primary tumor size and percent of lymph node occupied by infiltrating tumor, and second-ALN series 1 CD4 T cell, CD1a dendritic cell and the ratio of CD4:CD8 T cell populations in predicting disease-free survival. ALN series 1 represent the first, arbitrarily selected ALN per subject in the learning set. Greater area under the curve indicates greater predictive strength. Adjusted P values were determined by ROC curve testing for comparison of variable's predictive capacity.

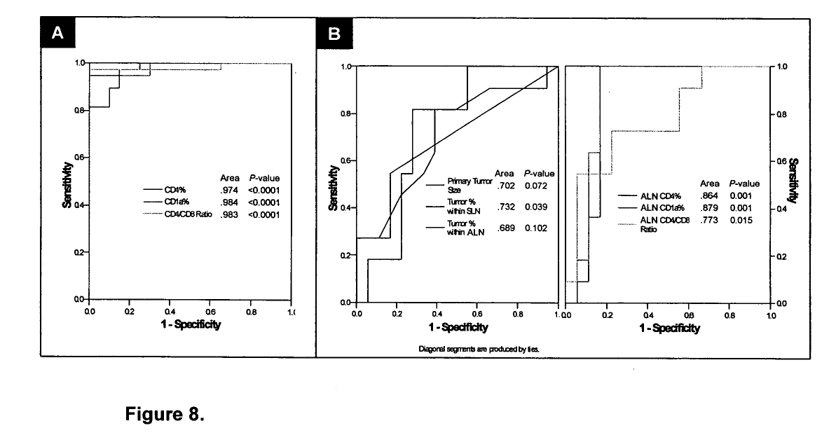

FIGS. 9A-9D. T Cell Interactions with Lymph Node-Infiltrating Breast Cancer. Architectural pattern analysis of T cell location delineated unique cell distribution for CD4 and CD8 T cells relative to the infiltrating focus of tumor. CD4 T cells were not preferentially located at the tumor border (Panel A) or within the intranodal tumor focus. CD8 T cells however were significantly denser at the tumor border (Panel B) and within the tumor focus (Panels C and D) compared to CD8 T cell density in the entire lymph node. All images were stained by immunohistochemistry with AE-1/AE-3 for breast cancer cells and labeled with VIP (purple). Double-stains with CD4 (Panel A) or CD8 (Panels B-D) were labeled with DAB (brown). Representative images were acquired at 100×, shown with variable zoom (Panels A-C), and 400× (Panel D).

FIG. 10. Architectural Pattern Analysis Model of Lymphocyte-Tumor Interactions. Tumor-involved lymph node imaging for CD4 and CD8 T cell populations, as well as granzyme B and CD25 staining, was performed by subdivision of the entire lymph node into four regions, allowing characterization of changes in lymphocyte populations based on distance from the infiltrating tumor cells. Each nodal area was partitioned and analyzed for cell type and population with areas defined as, I. entire nodal surface area—all stained areas of the node (includes areas I through IV); II. tumor margin—a rim of one medium-powered field (100×) around the tumor focus; III. tumor border—a series of contiguous high-powered fields (200×) with the center of each image at the interface of infiltrating tumor and nodal lymphocytes, circling the tumor focus; and, IV. tumor focus—all areas within the tumor border. Lymphocyte populations were calculated as percent lymph node surface area with a correction for the area occupied by tumor cells.

FIGS. 11A-11D. CD4 and CD8 T Cell Architectural Pattern Analysis. (A) CD8 T cell staining of 16 tumor-involved lymph nodes with analysis by lymph node region provided quantified values of CD8 T cell populations within regions I-IV (I. entire lymph node, II. tumor margin, III. tumor border, IV. tumor focus). Populations are expressed as percent of lymph node surface area, with mean and standard error of the mean displayed. Comparison of populations was performed by analysis of variance with Tukey's honestly significantly different test (†p=0.011,*p=0.004). (B) CD4 T cell staining of 16 tumor-involved lymph nodes with analysis by lymph node region provided quantified values of CD4 T cell populations within regions I-IV (I. entire lymph node, II. tumor margin, III. tumor border, IV. tumor focus). Populations are expressed as percent of lymph node surface area, with mean and standard error of the mean displayed. Comparison of populations was performed by analysis of variance with Tukey's honestly significantly different test (p>0.05 for all comparisons). (C) Granzyme B staining of 16 tumor-involved lymph nodes and image comparison to CD8 T cell staining with analysis by lymph node region provided quantified values of granzyme B expression by CD8 T cells within regions I-IV (I. entire lymph node, II. tumor margin, III. tumor border, IV. tumor focus). Populations are expressed as percent of lymph node surface area, with mean and standard error of the mean displayed. Comparison of granzyme B expression between regions was performed by analysis of variance with Tukey's honestly significantly different test (*p=0.001, †p=0.001, ‡p=0.001). Percent of CD8 T cells expressing granzyme B is noted by region. Comparisons of total CD8 T cell population versus CD8 T cell population staining with granzyme B within each region were performed by Wilcoxon rank sum tests with correction for multiple comparisons (§p=0.032, ∫ ∫ p<0.001). (D) CD25 staining of 16 tumor-involved lymph nodes and image comparison to CD4 T cell staining with analysis by lymph node region provided quantified values of CD25^{high} expression by CD4 T cells within regions I-IV (I. entire lymph node, II. tumor margin, III. tumor border, IV. tumor focus). Populations are expressed as percent of lymph node surface area, with mean and standard error of the mean displayed. Comparison of CD25^{high} expression between regions was performed by analysis of variance with Tukey's honestly significantly different test (*p=0.030, †p=0.071). Percent of CD4 T cells expressing CD25^{high} is noted by region. Comparisons of total CD8 T cell population versus CD8 T cell population staining with CD25^{high} within each region were performed by Wilcoxon rank sum tests with correction for multiple comparisons (§p=0.035).

FIGS. 12A-12C. ALN FOXP3 Expression by Nodal and Disease Status. (A) FOXP3 staining of 10 control lymph nodes and 2 ALNs per patient was stratified by nodal status, 38 of 58 ALNs were tumor-involved, and patient's disease status, with 11 of 29 patients developing breast cancer recurrence within 5 years. Of 20 tumor-free ALNs (TFLN), 5 were from patients with recurrent disease. Of 38 tumor-involved ALNs (TILN), 17 were from patients with recurrent disease. Populations staining FOXP3 are expressed as percent of lymph node surface area, with mean and standard error of the mean displayed. Comparison of FOXP3 expression within control lymph nodes, TFLNs and TILNs from breast cancer patients was performed by analysis of variance with Tukey's honestly significantly different test (*p=0.027). (B) Relative FOXP3 expression (ratio of percent of lymph node surface area staining FOXP3+ to percent CD4+) compared between control lymph nodes, TFLNs, and TILNs was performed by analysis of variance with Tukey's honestly significantly different test (*p<0.001, †p<0.001). (C) Comparison of relative FOXP3 expression within control lymph nodes, TFLNs and TILNs from patients with and without disease recurrence at five years was performed by analysis of variance with Tukey's honestly significantly different test (*p=0.002).

FIGS. 13A-13B. ALN Dendritic Cell Maturation Profile by Nodal and Disease Status. (A) CD83 and CD1a staining of 10 control lymph nodes and 2 ALNs per patient was stratified by nodal status, 38 of 58 ALNs were tumor-involved, and patient's disease status, with 11 of 29 patients developing breast cancer recurrence within 5 years. Of 20 tumor-free ALNs (TFLN), 5 were from patients with recurrent disease. Of 38 tumor-involved ALNs (TILN), 17 were from patients with recurrent disease. Dendritic cell maturation status was calculated as the ratio of percent of lymph node surface area staining with CD83 (a mature dendritic cell phenotypic marker) to percent of lymph node surface area staining with CD1a (present on both immature and mature dendritic cells), with mean and standard error of the mean displayed. Comparison of dendritic cell maturation within control lymph nodes and both tumor-free and tumor-involved nodes was performed by analysis of variance with Tukey's honestly significantly different test (no statistically significant differences between nodal groups observed). (B) Comparison of dendritic cell maturation within control nodes, tumor-free and tumor-involved nodes from patients with and without breast cancer recurrence was performed by analysis of variance with Tukey's honestly significantly different test (*p=0.036).

### DETAILED DESCRIPTION OF THE EMBODIMENTS

Cancers, particularly solid tumors, e.g. carcinoma, melanoma, etc. are phenotyped by analysis of the presence of immune cells, particularly T cells and dendritic cells, that are differentially present in regional lymph nodes, as compared to a control lymph node sample. Such phenotyping is useful for prognosis and treatment.

Lymph node samples containing white blood cells are stained with reagents specific for markers present on T cells and dendritic cells. The reagents, e.g. antibodies, may be detectably labeled, or may be indirectly labeled in the staining procedure. The data provided herein demonstrate that the distribution of these cells in regional lymph nodes associated with solid cancers provides for patient prognosis.

When non-sentinel regional lymph nodes of a cancer patient are compared to normal lymph nodes, it is shown herein that increased number of dendritic cells and decreased numbers of T cells, particularly T helper cells, correlate with a positive prognosis and disease-free survival following conventional chemotherapy. In such patients, conventional therapy may be sufficient for treatment. In patients that lack these characteristics, in which there is a negative prognosis, it may be desirable to provide more aggressive treatment at an early stage.

Any combination of markers may be used that are sufficient to distinguish the cells of interest. A marker combination of interest may include CD4 and CD1a, which distinguishes helper T cells and dendritic cells. CD8 may be included to differentiate cytolytic T cells. Alternatively, the T cell compartment may be analyzed by staining for CD3; CD45; CD28; CD25, granzymes B, FoxP3, etc. The information thus derived is useful in prognosis and diagnosis. Where the prognosis is, including susceptibility to acceleration of disease, status of a diseased state and response to changes in the environment, such as the passage of time, treatment with drugs or other modalities.

As used herein, the term “dendritic cell” refers to any member of a diverse population of morphologically similar cell types found in lymphoid or non-lymphoid tissues. Dendritic cells are referred to as “professional” antigen presenting cells, and have a high capacity for sensitizing MHC-restricted T cells. Dendritic cells may be recognized by function, by phenotype and/or by gene expression pattern, particularly by cell surface phenotype. These cells are characterized by their distinctive morphology, high levels of surface MHC-class II expression and ability to present antigen to CD4⁺ and/or CD8⁺ T cells, particularly to naïve T cells (Steinman et al. (1991) Ann. Rev. Immunol. 9:271; incorporated herein by reference for its description of such cells).

The cell surface of dendritic cells is unusual, with characteristic veil-like projections, and is characterized by expression of the cell surface markers CD11c and MHC class 11. Most DCs are negative for markers of other leukocyte lineages, including T cells, B cells, monocytes/macrophages, and granulocytes. Subpopulations of dendritic cells may also express additional markers including 33D1, CCR1, CCR2, CCR4, CCR5, CCR6, CCR7, CD1a-e, CD4, CD5, CD8alpha, CD9, CD11b, CD24, CD40, CD48, CD54, CD58, CD80, CD83, CD86, CD91, CD117, CD123 (IL3Rα), CD134, CD137, CD150, CD153, CD162, CXCR1, CXCR2, CXCR4, DCIR, DC-LAMP, DC-SIGN, DEC205, E-cadherin, Langerin, Mannose receptor, MARCO, TLR2, TLR3 TLR4, TLR5, TLR6, TLR9, and several lectins. The use of CD1as a marker is of particular interest, including CD1a. The patterns of expression of these cell surface markers may vary along with the maturity of the dendritic cells, their tissue of origin, and/or their species of origin.

T cells, or T lymphocytes, are small mononuclear cells of the immune system. They are characterized by expression of the T cell antigen receptor, which is associated with the invariant CD3 chain. Mature T cells may be broadly divided into CD4⁺ cells, which primarily interact with Class II MHC molecules; and CD8⁺ cells, which primarily interact with Class I MHC molecules. Additional cell surface markers specific for T cells include CD25, CD45; CD28; FoxP3, granzymes B, and CD60c.

There is a relationship between the size of immature dendritic cell population and proportion of CD4⁺ lymphocytes expressing CD25^{high}, with additional supporting evidence by FoxP3 (a marker for T regulatory cells) within axillary nodes of patients with breast cancer. A clinical outcome benefit, specifically disease-free survival, is found among breast cancer patients with decreased populations of T regulatory cells. The perturbations of T lymphocyte and dendritic cell populations, architecturally as related to the location of metastatic tumor cells, and phenotypically through characterization of CD25, FoxP3, granzyme B, and dendritic cell maturation, provide clinically-based support for the progression of tumor dependent immune modulation. Lymph node T regulatory cell population size and dendritic cell maturation predict risk of breast cancer recurrence.

The subject methods are useful in the analysis of cancer. Cancer cells are characterized by uncontrolled growth, invasion to surrounding tissues, and metastatic spread to distant sites. It is well known in the art that cancers, particularly cancers that form solid tumors, will invade the lymph nodes near the area of the primary lesion, and it is common in the assessment of many different cancers to biopsy such lymph nodes to search for the presence of micrometastases. The specific lymph nodes involved will depend on the site of the tumor. For example, breast cancer involves the axillary lymph nodes. The regional lymph nodes for cancers such as melanoma will depend on the placement of the initial tumor in the body.

In addition to the analysis of regional nodes, it is also known in the art that the “sentinel node”, which is the lymph node into which the tumor initially drains, can be identified by the use of various tracers, and provides additional insight as to the presence of metastases. Sentinel lymph nodes have been identified in melanoma (Morton et al. (1992) Arch Surg. 127(4):392-9); gastric cancer (Moenig et al. (2005) Anticancer Res. 25(2B):1349-52); head and neck squamous cell carcinoma (Kovacs et al. (2005) Otolaryngol Head Neck Surg. 132(4):570-6); cervical and endometrial cancer (Holub et al. (2004) Med Sci Monit. 10(10):CR587-91); non-small cell lung cancer (Minamiya et al. (2005) Ann Thorac Cardiovasc Surg. 11(2):67-72); colorectal cancer (Dahl et al. (2005) Eur J Surg Oncol. 31(4):381-5); and the like.

Tumors of interest for treatment include melanomas, carcinomas, such as squamous cell carcinomas, adenocarcinomas, transitional cell carcinomas, basal cell carcinomas, etc., which may include colon, duodenal, prostate, breast, melanoma, ductal, hepatic, pancreatic, renal, endometrial, stomach, dysplastic oral mucosa, polyposis, invasive oral cancer, non-small cell lung carcinoma, transitional and squamous cell urinary carcinoma, etc.

Some cancers of particular interest include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Also of interest is non-small cell lung carcinoma. Non-small cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

### Differential Cell Analysis

Analysis of cancer associated lymph node samples allows determination of the stage of the cancer, including prognosis of breast cancer. Clinical samples for use in the methods of the invention are typically obtained from biopsy of lymph nodes associated with a cancer of interest, although in some embodiments the analysis is performed in vivo. Samples can be separated by centrifugation, elutriation, density gradient separation, apheresis, affinity selection, panning, FACS, centrifugation with Hypaque, etc. prior to analysis, and usually a mononuclear fraction (PBMC) will be used. Once a sample is obtained, it can be used directly, frozen, or maintained in appropriate culture medium for short periods of time. Various media can be employed to maintain cells. The samples may be obtained by any convenient procedure. Usually a sample will comprise at least about 10² cells, more usually at least about 10³ cells, and preferable 10⁴, 10⁵ or more cells. Typically the samples will be from human patients, although animal models may find use, e.g. equine, bovine, porcine, canine, feline, rodent, e.g. mice, rats, hamster, primate, etc.

An appropriate solution may be used for dispersion or suspension of the cell sample. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate-buffers, lactate buffers, etc.

Analysis of the cell staining will use conventional methods. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The labeled cells are then quantitated as to the expression of cell surface markers as previously described. In a clinical setting, an immunoassay may be performed in a self-contained apparatus. A number of such methods are known in the art. The apparatus will generally employ a continuous flow-path of a suitable filter or membrane, having at least three regions, a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The measuring region may have bound to it a conjugate of an enzyme with cell specific antibodies.

Alternatively, a number of methods are known in the art for quantitating cells bound marker specific antibodies, including immunohistochemistry of slides; fluorescent microscopy; flow cytometry; and the like.

Where the analysis is performed in vivo, labeled antibodies are injected into the region of the tumor, e.g. in the axilla, and the signals measured over time to determine specific binding. Preferred radiographic moieties for use as imaging moieties include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Examples of such compositions, which may be utilized for x-ray radiography are described in U.S. Pat. No. 5,709,846, incorporated fully herein by reference. Such moieties may be conjugated to the antibody through an acceptable chemical linker or chelation carrier. In addition, radionuclides that emit radiation capable of penetrating the skull may be useful for scintillation imaging techniques. Suitable radionuclides for conjugation include ⁹⁹Tc, ¹¹¹In, and ⁶⁷Ga. Positron emitting moieties for use in the invention include ¹⁸F, which can be easily conjugated by a fluorination reaction with the antibody according to the method described in U.S. Pat. No. 6,187,284.

Preferred magnetic resonance contrast moieties include chelates of chromium(III), manganese(II), iron(II), nickel(II), copper(II), praseodymium(III), neodymium(III), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates may be conjugated to the antibodies through a suitable chemical linker.

Optically visible moieties for use as imaging moieties include fluorescent dyes, or visible-spectrum dyes, visible particles, and other visible labeling moieties. Fluorescent dyes such as fluorescein, coumarin, rhodamine, bodipy Texas red, and cyanine dyes, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes include FDA-approved food dyes and colors, which are non-toxic, although pharmaceutically acceptable dyes which have been approved for internal administration are preferred. In preferred embodiments, such dyes are encapsulated in carrier moieties, which are in turn conjugated to the antibody. Alternatively, visible particles, such as colloidal gold particles or latex particles, may be coupled to the antibody moiety via a suitable chemical linker.

The comparison of a differential immune profile obtained from a patient sample, and a reference immune profile is accomplished by the use of suitable deduction protocols, Al systems, statistical comparisons, etc. A comparison with a reference immune profile from normal lymph node cells, cells from similarly diseased tissue, and the like, can provide an indication of the disease staging. A database of reference immune profiles can be compiled. The methods of the invention provide an early prognostic evaluation, and therefore allow early therapeutic intervention, e.g. initiation of chemotherapy, increase of chemotherapy dose, changing selection of chemotherapeutic drug, and the like.

The data may be subjected to non-supervised hierarchical clustering to reveal relationships among profiles. For example, hierarchical clustering may be performed, where the Pearson correlation is employed as the clustering metric. One approach is to consider a patient immune profile as a “learning sample” in a problem of “supervised learning”. CART is a standard in applications to medicine (Singer (1999) Recursive Partitioning in the Health Sciences, Springer), which may be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

This approach has led to what is termed FlexTree (Huang (2004) PNAS 101:10529-10534). FlexTree has performed very well in simulations and when applied to SNP and other forms of data. Software automating FlexTree has been developed. Alternatively LARTree or LART may be used Fortunately, recent efforts have led to the development of such an approach, termed LARTree (or simply LART) Turnbull (2005) Classification Trees with Subset Analysis Selection by the Lasso, Stanford University. The name reflects binary trees, as in CART and FlexTree; the lasso, as has been noted; and the implementation of the lasso through what is termed LARS by Efron et al. (2004) Annals of Statistics 32:407-451. See, also, Huang et al. (2004) Tree-structured supervised learning and the genetics of hypertension. Proc Natl Acad Sci USA. 101 (29):10529-34.

Other methods of analysis that may be used include logic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as PAM software and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome.

Also provided are databases of immune profiles. Such databases will typically comprise immune profiles of individuals and of reference, or normal samples, where such profiles are as described above.

The analysis and database storage may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The immune profiles and databases thereof may be provided in a variety of media to facilitate their use. “Media” refers to a manufacture that contains the immune profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

Kits may be provided, where the kit will comprise a staining reagents that are sufficient to differentially identify the immune cells described herein. A marker combination of interest may include a T cell specific marker, and a dendritic cell specific marker. For example, detectable labeled or unlabeled reagents specific for CD1a and CD4 may be provided. The staining reagents are preferably antibodies. Kits may also include tubes, buffers, etc., and instructions for use.

It is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

The following examples are offered by way of illustration and not by way of limitation.

### EXPERIMENTAL

### EXAMPLE 1

Methods

STUDY PATIENTS. Breast cancer patients aged 29-80 years treated at Stanford University Medical Center between February 1997 and January 1999 and found to have tumor-involved SLNs by multilevel hematoxylin and eosin staining (HES) or immunohistochemistry (IHC) were evaluated. Patients who subsequently underwent ALN dissection, as is standard clinical practice, with clinical outcome data available were selected. SLNs and ALNs were selected based on their designation as sentinel or axillary by the operative report. In surgical cases involving multiple SLNs and ALNs, one SLN (SLN series 1) and one ALN (ALN series 1) were arbitrarily selected by the Department of Pathology staff and represent the training set, n=29. The Pathology staff member was blinded to the study design. As no randomization technique was employed the training set selection process is by definition arbitrary rather than random. To test reliability and variance of immune profile, eight ALNs were selected from a single patient. For purposes of validating the training set, first, a second SLN and ALN were randomly selected for each subject within the training set and represent training set SLN series 2, n=18 (11 of 29 patients had only a single SLN removed which was included in SLN series 1), and training set ALN series 2, n=27 (an additional ALN could not be retrieved for 2 of the original 29 patients). Second, a single ALN was randomly selected for all subjects within the test set (n=48). SLNs and ALNs from patients within training set SLN series 2, training set ALN series 2, and the test set were randomly selected using random number assignment software with R. As performed in prior studies to provide an average immune profile, ten control nodes, a single mesenteric node per control subject, were similarly examined from patients with benign disease without a history of malignancy or immune disorder. All samples were collected from Stanford Department of Pathology Specimen Bank as coded specimens under a protocol approved by the Stanford University Medical Center Institutional Review Board.

All subjects were untreated and without a history of cancer or immune disorder prior to breast cancer diagnosis and SLN biopsy. Following surgical management, patients received adjuvant therapy as determined by their medical and radiation oncologists. The duration of disease-free survival was the time between initial diagnosis and first recurrence. All patients received SLN and ALN removal in conjunction with removal of primary tumor within 44 days of initial diagnosis. Initial diagnosis was performed by needle aspiration or core biopsy in the majority of cases. Final diagnosis was confirmed from the pathologic evaluation of the primary tumor from the lumpectomy specimen. The average difference between time of diagnosis and surgery was 12.3 days. We chose to use time of diagnosis rather than time of surgery to determine clinical outcome as we are measuring the interaction between tumor and immune composition of local nodes versus the influence of surgery on outcome. All recurrences were based on documentation of local or systemic disease during a follow-up period of five years after which data were censored. We recorded and verified patient, tumor, and lymph node characteristics.

PATIENT, PRIMARY TUMOR, AND LYMPH NODE CHARACTERISTICS. Characteristics of the training set are shown in Table 1. Of 29 SLN metastases in SLN series 1, all were tumor-involved, 5 contained isolated tumor cells, 11 micrometastases, and 13 contained macrometastases. Of 18 SLNs in series 2, 9 were tumor-involved, 3 contained micrometastases and 6 macrometastases. 16 subjects had positive ALN dissections. 9 of 29 arbitrarily selected series 1 ALNs were found to be tumor-involved with 7 of the 20 tumor-free ALNs selected from patients with positive ALN dissections (ALNs other than the one selected for series 1 were found to be tumor-involved)(FIG. S1). 7 of 27 randomly selected series 2 ALNs were tumor-involved (FIG. S2). 11 of 29 patients developed recurrent disease with 60 months of follow-up. 2 of 11 recurrences (18%) occurred at a distant site, and 10 of 11 developed locoregional relapse (91%), with one patient at time of relapse found to have both local and distant disease.

TABLE 1
Patient, Primary Tumor and Lymph
Node Characteristics

| | | | Test Set | |
|---|---|---|---|---|
| | Training Set | | Patients, | |
| | Patients, | | number | |
| Characteristic | number (n = 29) | % | (n = 48) | % |
| Patient Characteristics | | | | |
| Age (years) | 52 (29-76) | | 52 (33-80) | |
| Age < 51 | 16 | 55.2 | 23 | 47.9 |
| Age = 51 | 13 | 44.8 | 25 | 52.1 |
| Stage | | | | |
| Stage IIA | 11 | 37.9 | 20 | 41.7 |
| Stage IIB | 11 | 37.9 | 16 | 33.3 |
| Stage IIIA | 7 | 24.1 | 11 | 22.9 |
| Stage IIIB | 0 | 0 | 0 | 0 |
| Stage IIIC | 0 | 0 | 1 | 2.1 |
| Primary Tumor Characteristics | | | | |
| Tumor size (mm) | 21.4 (2.0-75) | | 25.3 (7.0-90) | |
| Histological tumor stage | | | | |
| T1a | 1 | 3.4 | 0 | 0 |
| T1b | 4 | 13.8 | 2 | 4.2 |
| T1c | 11 | 37.9 | 23 | 47.9 |
| T2 | 11 | 37.9 | 22 | 45.8 |
| T3 | 2 | 6.9 | 1 | 2.1 |
| Tumor grade | | | | |
| G1: Low combined histologic grade | 2 | 6.9 | 2 | 4.2 |
| G2: Intermediate combined histologic grade | 17 | 58.6 | 29 | 60.4 |
| G3: High combined histologic grade | 10 | 34.5 | 17 | 35.4 |
| ER status | | | | |
| Negative | 12 | 41.4 | 4 | 8.3 |
| Positive | 17 | 58.6 | 39 | 81.3 |
| PR status | | | | |
| Negative | 13 | 44.8 | 9 | 18.8 |
| Positive | 16 | 55.2 | 34 | 70.8 |
| HER2/neu expression | | | | |
| Not overexpressed | 18 | 62.1 | 22 | 45.8 |
| Equivocal | 3 | 10.3 | 3 | 6.3 |
| Overexpressed | 8 | 27.6 | 12 | 25.0 |
| Angiolymphatic invasion | | | | |
| None | 19 | 65.5 | 12 | 25.0 |
| Present | 10 | 34.5 | 20 | 41.7 |
| Sentinel Lymph Node Characteristics | | | | |
| Number of tumor-free sentinel lymph nodes | 0.86 (0-4) | | 0.44 (0-3) | |
| 0 | 16 | 55.2 | 34 | 70.8 |
| =1 | 13 | 44.8 | 14 | 29.2 |
| Size of SLN metastases | | | | |
| Isolated tumor cells = 0.2 mm | 5 | 17.2 | 4 | 8.3 |
| Micrometastases = 2 mm | 11 | 37.9 | 24 | 50.0 |
| Macrometastases > 2 mm | 13 | 44.8 | 20 | 41.7 |
| Percent SLN infiltrated by tumor§ | 1.23 (0.01-84.0) | | 1.35 (0.01-89.2) | |
| <1.5% | 16 | 55.2 | 24 | 50.0 |
| =1.5% | 13 | 44.8 | 24 | 50.0 |
| Extracapsular extension | | | | |
| None | 26 | 89.7 | 28 | 58.3 |
| Present | 3 | 10.3 | 7 | 14.6 |
| Sentinel lymph node metastases identification | | | | |
| Hematoxylin and eosin staining | 19 | 65.5 | 43 | 89.6 |
| Immunohistochemistry | 10 | 34.5 | 5 | 10.4 |
| Nodal stage | | | | |
| N1 | 23 | 79.3 | 37 | 77.1 |
| N2 | 6 | 20.7 | 10 | 20.8 |
| N3 | 0 | 0 | 1 | 2 |
| Axillary Lymph Node Characteristics | | | | |
| Palpable axilllary lymph node | | | | |
| None | 20 | 69.0 | 30 | 62.5 |
| Present | 9 | 31.0 | 18 | 37.5 |
| Number of tumor-free axillary (nonsentinel) | 6.72 (1-19) | | 9.42 (0-27) | |
| lymph nodes | | | | |
| <7 | 16 | 55.2 | 22 | 45.8 |
| =7 | 13 | 44.8 | 26 | 54.2 |
| Number of tumor-involved axillary (nonsentinel) | 1.72 (0-11) | | 1.35 (0-9) | |
| lymph nodes | | | | |
| 0 | 13 | 44.8 | 22 | 45.8 |
| =1 | 16 | 56.2 | 26 | 54.2 |
| Axillary lymph node metastases identification* | | | | |
| None | 13 | 44.8 | 17 | 35.4 |
| Hematoxylin and eosin staining | 11 | 37.9 | 22 | 45.8 |
| Immunohistochemistry | 5 | 17.2 | 9 | 18.8 |

*Presence of tumor-involvement within axillary lymph nodes independent of SLN status (allSLNs are tumor-involved).
†Recurrence during follow-up of 60 months.
‡Variables not significant at P < 0.1. Multivariate analysis by logistic regression was not performed on non-significant variables by univariate analyses.
§Percent of node involved by tumor was determined by immunohistochemical analysis.

Test set clinicopathologic characteristics are shown in Table 1. All patients had a tumor-involved SLN biopsy, with 4 containing isolated tumor cells, 24 micrometastases, and 20 containing SLN macrometastases. 22 of 48 patients (45.8%) developed recurrent disease during follow-up of 60 months. 14 of 22 occurred at distant sites, 7 developed locoregional relapse, and one recurred both at a distant site and locally. ALNs selected from 8 of 22 patients, 36.3%, with disease recurrence were tumor-involved (FIG. S3). Of the 26 ALNs selected from patients without recurrent disease, 9 were tumor-involved, 34.6%.

Among all ALNs from both training set and test (n=104), only tumor size significantly correlated with disease recurrence (P=0.015). Among ALNs selected from patients with only T1 (n=56), median number of tumor-involved ALNs correlated with disease recurrence superior to all other clinicopathologic characteristics, (P=0.070). Likewise, among ALNs selected from patients with only T2 tumors (n=43), size of SLN metastasis correlated with disease recurrence more closely than all other clinicopathologic characteristics (P=0.100).

IMMUNOSTAINING. Tissue sections, 3 μm thick, were cut from formalin-fixed, paraffin-embedded nodes. HES and IHC were performed after antigen retrieval using Biogenex Genomx i1000 (San Ramon, Calif.). Antibodies used included anti-CD4 (1/20, Novacastro), anti-CD8 (1/25, Dako), anti-CD1a (1/100, Dako), anti-AE1/AE3 (1/25, Biogenex), and as secondary antibody—EnVision dextran kit (1/5, Dako). Optimal concentrations were determined, and tested in sample node sections. Double staining using 3′3′ diaminobenzidene, VIP (Vector Laboratories), and a light counterstain with Mayer's hematoxylin (Innogenex) was performed for lymphocyte population of interest with and colocalization of tumor cells. Isotype-matched antibodies were used as negative controls. All slides for the respective antibody were stained in the same run.

Presence of metastasis was verified by HES and IHC on four sections per node by two blinded investigators trained in breast cancer pathology. Area of node occupied by each immune cell type and by tumor was determined through computerized image acquisition and analysis software, BLISS (Bacus Lab Inc., Slide Scanner). Prior image analyses determine cell count and area from an average of five to 20 high-power fields [10,14,15,20,25]. Using BLISS we acquired 160-4130 sequential images at 200× of the entire lymph node section, which were sequenced together by Metamorph Imaging System (Universal Imaging). Objectives were calibrated to transform image pixels to μm. Control nodes were examined to standardize thresholds of each stain for cell of interest. Using an automated Metamorph script, standardized thresholds were applied with Metamorph log set to record areas occupied by cell of interest, tumor, and of entire node for all samples, thus minimizing any potential operator bias.

STATISTICAL ANALYSIS. Univariate and multivariate analyses including logistic regression tested predictive capacity of patient characteristics. Immune profiles of patients with and without nodal metastasis, and with and without disease recurrence were compared by Wilcoxon rank sum test. F-test for immune profile equality of variance analysis was used to determine variance between nodes from a single patient versus nodes from different patients with similar characteristics. ALN series 1 immune profile's sensitivity and specificity in predicting disease recurrence were determined from receiver-operating-characteristic (ROC) curves based on the ALN immune profile of patients with versus without disease recurrence from the training set. ALN series 1 immune profile thresholds were applied to SLN series 2, ALN series 2 and the test set with statistical comparison by X² test and calculation of the correlation coefficient. We constructed Kaplan-Meier (KM) life-table curves for disease-free survival, with permuted log-rank test comparisons as the sample size was limited. The training set was stratified for KM curves by ALN series 1 and 2 immune profiles, established from ROC curves applied to ALN series 1, to test prediction of disease-free survival. Nodal thresholds from the training set ALN series 1 were also applied to the test set in KM curves compared by permuted log-rank tests. Two-sided P<0.05 corrected for multiple comparisons was considered a statistically significant difference. For analyses we used SPSS software and R statistical package.

Results

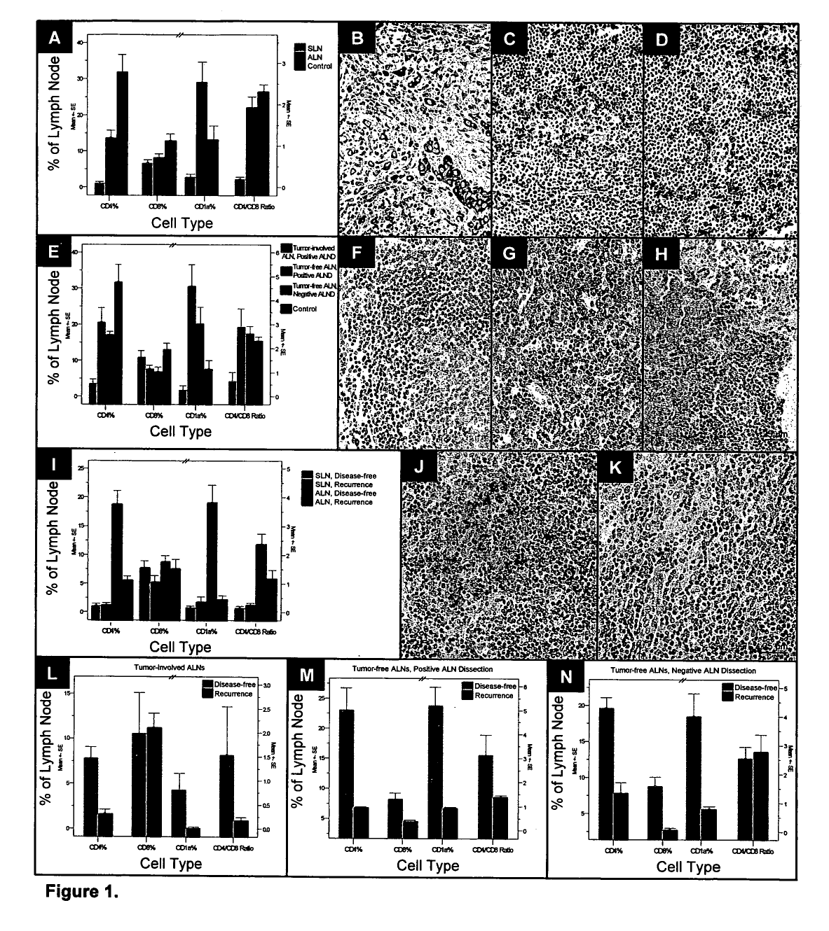

Alterations in Immune Profile of Tumor-Draining Lymph Nodes. To determine whether tumor-draining lymph nodes from patients with breast cancer are different immunologically than lymph nodes from control individuals, we initially analyzed one SLN and one ALN from each of 29 breast cancer patients (training set, Table 1) by IHC for CD4 T cell, CD8 T cell, and CD1a dendritic cell populations (FIG. 1). We found significant differences in CD4 and CD1a populations between SLN, ALN, and control nodes (FIG. 1A). While control nodes contained the highest percentages of CD4 and CD8 T cells, ALNs contained the highest percentage of CD1a cells (FIG. 1A). The magnitude of CD4 population decrease from control nodes to SLNs was over 10-fold greater than the CD8 decreases between these nodes. SLNs also displayed significant decreases in CD1a cells. Interestingly, CD1a cells were elevated in ALNs even above controls. To determine if tumor invasion is a prerequisite for alterations in immune profile, training set SLNs and ALNs were grouped together as tumor-free or tumor-involved, which revealed dramatic differences in CD4 and CD1a populations and CD4:CD8 ratio based on tumor status (Table 2). Furthermore, training set ALNs (FIG. S1) were stratified as tumor-involved (n=9), tumor-free from an individual with positive ALND (n¼7), or tumor-free from an individual with negative ALND (n=13). CD4 and CD1a cells were significantly decreased in tumor-involved ALNs (FIG. 1E). Intriguingly, CD4 populations were decreased even in tumor-free ALNs (FIG. 1E), suggesting that these changes are not merely a reflection of tumor invasion. In contrast, tumor free ALNs showed significant increases in CD1a cells, which is more dramatic in those from individuals with a positive ALND (FIG. 1E). Analysis of percent of node involved by tumor and magnitude of CD4, CD8, or CD1a changes did not show a statistically significant relationship. These observations argue against a simple linear relationship between immune alterations and tumor invasion, but suggest that dynamic changes in the immune profile within tumor-draining lymph nodes may in fact precede tumor invasion.

TABLE 2
Immune Profile and Nodal Status

| | Tumor-free Lymph | Tumor-involved | | |
|---|---|---|---|---|
| | Node, % of lymph | Lymph Node, % of | Control Lymph Node,† | |
| | node (mean ± SE, | lymph node (mean ± SE, | % of lymph node | Wilcoxon rank sum test* |
| Cell Population | n = 20) | n = 38) | (mean ± SE, n = 10) | P-value |
| CD4 % | 17.85 ± 2.19 | 2.11 ± 0.35 | 31.93 ± 4.76 | <0.001 |
| CD8 % | 7.93 ± 0.99 | 7.52 ± 0.71 | 13.12 ± 1.71 | 0.890 |
| CD1a % | 3.59 ± 0.56 | 0.26 ± 0.06 | 1.17 ± 0.32 | <0.001 |
| CD4/CD8 Ratio | 2.47 ± 0.28 | 0.34 ± 0.08 | 2.33 ± 0.16 | <0.001 |

Training set analysis (29 series 1 sentinel lymph nodes, 29 series 1 axillary lymph nodes, total lymph nodes n = 58).
†Tumor-free control lymph nodes selected from 10 patients without cancer or immunodeficiency.
*Wilcoxon rank sum test for tumor-free lymph node immune profile versus tumor-involved lymph node immune profile.

Relationship between SLN Immune Profile and Axillary Metastasis or Disease-free Survival. We investigated if a relationship exists between SLN immune profile and ALN metastasis or disease-free survival. While SLN CD4 populations and CD4:CD8 ratio demonstrated a trend toward an association with axillary metastasis (Table 3), CD8 and CD1a populations showed no such relationship. When SLN immune profile was analyzed for DFS, CD8 populations showed a trend; however, all other cell populations showed no statistically significant relationship with survival (FIG. 1I; Table 3).

ALN Immune Profile and Disease-Free Survival. In contrast to SLNs, which exhibited similar immune profile changes in all 29 training set individuals, ALN CD4 and CD1a populations showed significant differences between patients with recurrence versus those disease-free at 5 y (p, 0.001) (FIGS. 1I and 1K; Table 4). Furthermore, associations between disease recurrence and changes in ALN CD4 and CD1a populations were independent of nodal metastasis or ALND status (FIGS. 1L-1N). Among patients with disease recurrence, degree of decrease in CD4 T cell and CD1a dendritic cell populations was similar (greater than 4-fold) among tumor-involved ALNs and tumor-free ALNs from either positive or negative ALNDs.

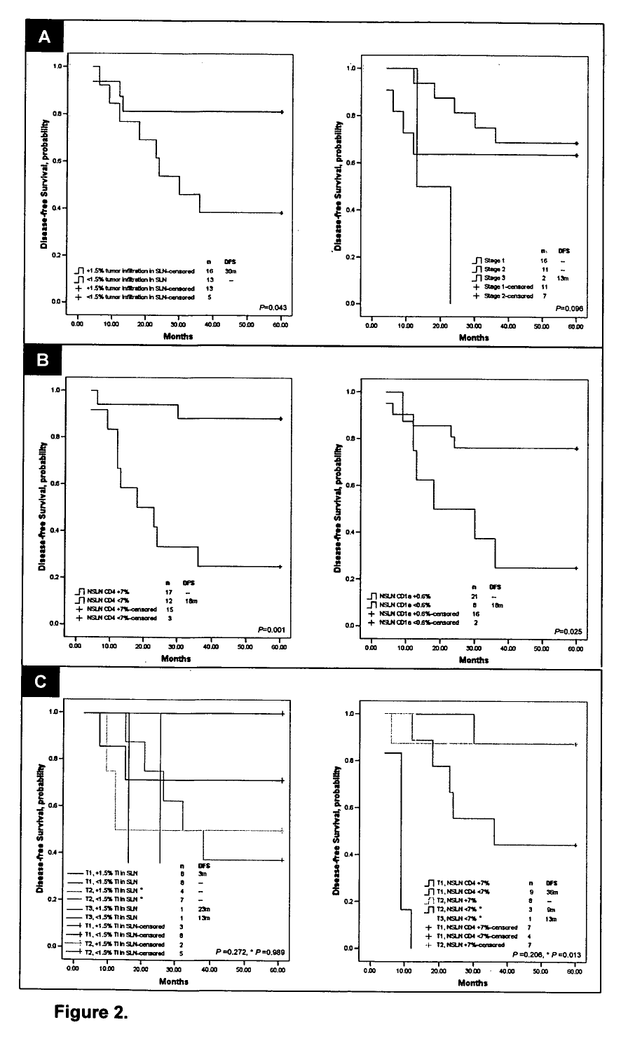

These findings support a direct relationship between ALN immune profile and disease-free survival—even within these arbitrarily selected ALNs (series 1), regardless of nodal and locoregional metastasis status. To expand on the applicability of these findings, we randomly selected a second ALN from 27 of the 29 individuals in the training set (series 2, FIG. S2). Immune profile thresholds determined from ROC curve analysis for maximal predictive accuracy among the training set ALN series 1 were applied to these additional 27 ALNs. Stratification of the training set into favorable and unfavorable prognostic groups for CD4 and CD1a populations was highly significant as displayed in KM curves of DFS (p=0.005 and p=0.007, respectively) (FIG. 2A; Table 4).

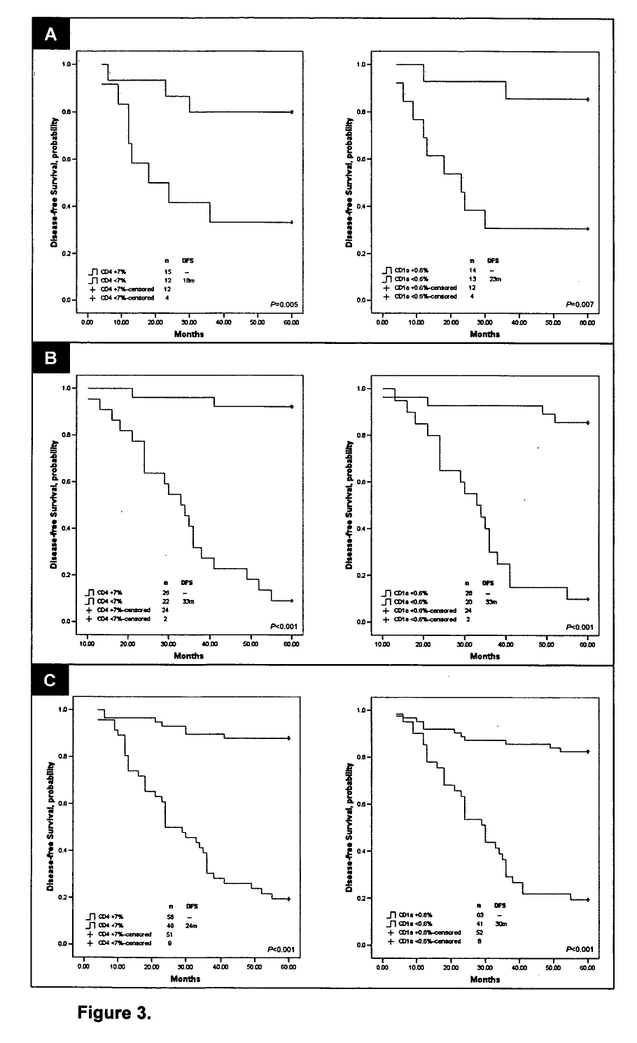

Additional comparison of immune profile and patient characteristics within the training set demonstrated ALN CD4 T cell and CD1a dendritic cell populations had superior predictive capacity of DFS (p=0.001 for both) compared to the degree of tumor involvement in SLNs and ALNs or to primary tumor size, by ROC curve analyses (p=0.039, p=0.102, and p=0.072) (FIG. S4). KM curves indicated significant stratification of DFS by percent of tumor involvement in SLN series 1, tumor stage, and ALN CD1a and CD4 populations (FIG. 3) (p=0.043, p=0.096, p=0.001, and p=0.025). Patient stratification by both ALN CD4 T cell population and tumor stage predicted DFS equally as well as, if not better than, the most statistically significant clinicopathologic characteristics (tumor stage and percent of tumor involvement in the SLN) (FIG. 3C).

TABLE 3
SLN Immune Profile and Clinical Outcome
Axillary Metastases

| | % of SLN of Patients | % of SLN of Patients | |
|---|---|---|---|
| | without Axillary | with Axillary | Wilcoxon rank sum |
| | Metastases, | Metastases, | test |
| Cell Population | (mean ± SE, n = 13) | (mean ± SE, n = 16) | P-value |
| CD4 % | 1.59 ± 0.38 | 1.00 ± 0.21 | 0.120 |
| CD8 % | 6.30 ± 0.76 | 7.40 ± 1.29 | 0.999 |
| CD1a % | 0.22 ± 0.07 | 0.28 ± 0.12 | 0.693 |
| CD4/CD8 Ratio | 0.28 ± 0.07 | 0.16 ± 0.11 | 0.101 |

Disease-free Survival

| | % of SLN of Patients | % of SLN of Patients | |
|---|---|---|---|
| | Disease-free at 60 | with Recurrent | Wilcoxon rank sum |
| | Months, | Disease at 60 Months, | test |
| Cell Population | (mean ± SE, n = 18) | (mean ± SE, n = 11) | P-value |
| CD4 % | 1.22 ± 0.30 | 1.34 ± 0.27 | 0.220 |
| CD8 % | 7.86 ± 1.08 | 5.36 ± 0.92 | 0.076 |
| CD1a % | 0.18 ± 0.04 | 0.38 ± 0.16 | 0.234 |

SLN, sentinel lymph node.

TABLE 4
ALN Immune Profile and Disease-free Survival

| | Immune Profile of | Immune Profile of Patients | |
|---|---|---|---|
| | Patients Disease-free, | with Recurrent Disease, | |
| | % of lymph node | % of lymph node | Wilcoxon rank sum test |
| Cell Population | (mean ± SE) | (mean ± SE) | P-value |
| Learning Set | | | |
| ALN Series 1 Immune Profile | | | |
| (n = 29)‡ | | | |
| CD4 % | 18.8 ± 2.35 | 5.62 ± 0.63 | <0.001 |
| CD8 % | 8.88 ± 0.96 | 7.66 ± 1.53 | 0.493 |
| CD1a % | 3.83 ± 0.59 | 0.47 ± 0.11 | <0.001 |
| ALN Series 2 Immune Profile | | | |
| (n = 27)* | | | |
| CD4 % | 18.17 ± 3.48 | 10.20 ± 3.77 | 0.026 |
| CD8 % | 9.57 ± 1.17 | 11.07 ± 3.22 | 0.512 |
| CD1a % | 2.42 ± 0.55 | 0.60 ± 0.22 | 0.015 |
| Test Set | | | |
| ALN Immune Profile | | | |
| (n = 48)† | | | |
| CD4 % | 26.57 ± 2.31 | 4.38 ± 1.42 | <0.001 |
| CD8 % | 17.88 ± 1.04 | 5.79 ± 0.89 | <0.001 |
| CD1a % | 3.02 ± 0.37 | 0.41 ± 0.13 | <0.001 |

‡ALN Series 1, 11 of 29 patients with recurrent disease with 60 months of follow-up.
*ALN Series 2, 11 of 27 patients with recurrent disease with 60 months of follow-up.
†Test Set, 22 of 48 subjects with recurrent disease with 60 months of follow-up.
ALN, axillary lymph node.

Intra-subject vs. Inter-subject Variance in Lymph Node Immune Profile. To more fully address the issue of inter-nodal variance in immune profile from a single subject, we analyzed the immune profiles of eight randomly-selected ALNs from a single patient. The variance of these nodes was compared to the variance of nodes from different subjects with similar patient characteristics, including similar recurrent disease state (n=66). Equality of variance testing illustrated intra-subject homogeneity between nodes relative to inter-subject nodal variance for CD1a, CD4, and CD8 (F(65,7)-statistics of 24.65, 26.89, and 10.23 corresponding significance P<0.001, P<0.001, and P=0.002, respectively).

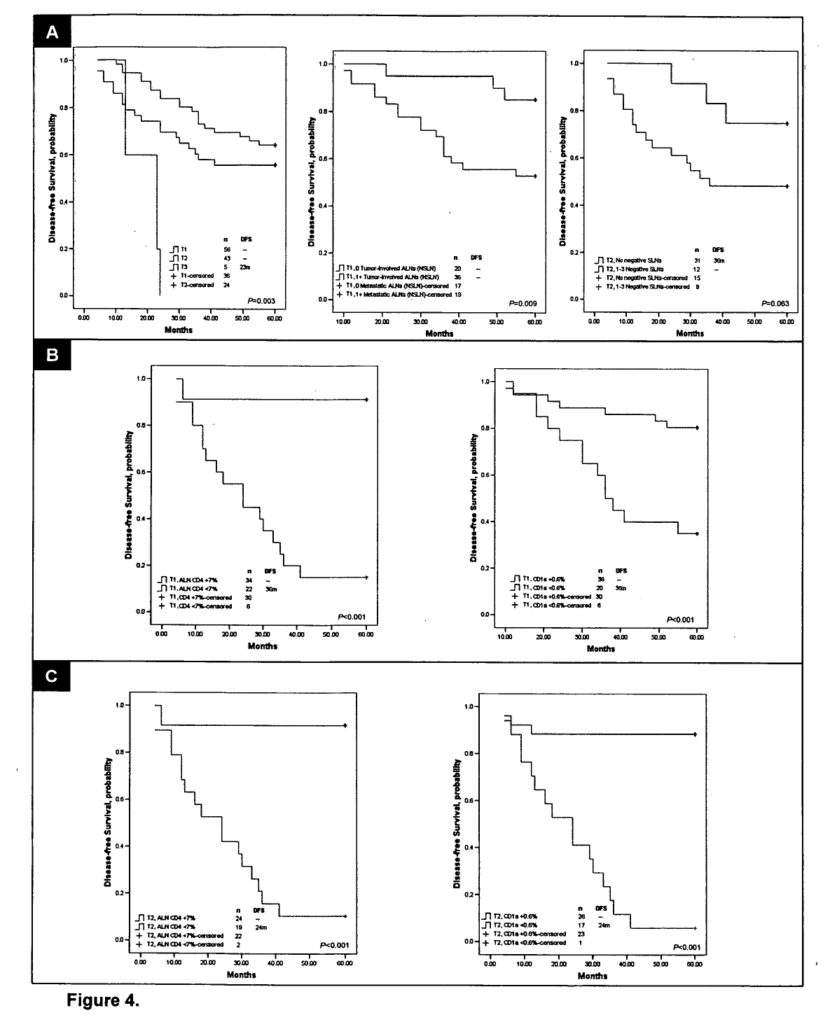

Validation of the Predictive Capacity of ALN Immune Profile. To further validate the predictive capacity of ALN immune profile for DFS in breast cancer, we analyzed one randomly selected ALN from an additional 48 patients (test set, Table 1), 22 of which developed recurrent disease in 5 y. Thresholds determined by ROC curves from the training set series 1 were applied to the test set data, which demonstrated highly significant stratification of favorable and unfavorable risk of recurrent disease (KM curves of DFS and permuted log-rank tests significant with p, 0.001 for both CD4 and CD1a populations; FIG. 2B). Final comparison of the predictive strength of ALN immune profile relative to the most predictive clinicopathologic characteristics was performed for all patients with recurrence status available (single ALN selected randomly from learning set series 1 or series 2, n=27; and ALN test set, n=48; total ALNs n=77; FIG. 2C). Of 77 patients analyzed, 33 developed recurrent disease during the follow-up period. Among all patients from both training set and test set, only tumor size significantly correlated with disease recurrence (p=0.015). KM curves of DFS stratified by ALN CD4 population and ALN CD1a population demonstrate superior risk stratification for recurrence by immune profiling compared to tumor size (p=0.001, p=0.001, and p=0.004, respectively; FIGS. 2C and 4A).

Strength of ALN Immune Profile as Predictors of Disease-Free Survival in Early Stage Patients (T1 and T2 Tumors). The predictive value of ALN immune profile was particularly striking in early stage breast cancer patients (with T1 and T2 tumors) (FIG. 4). Among the learning set, patients with T2 tumors and ALN CD4 population less than 7.0% had a median duration to recurrence of 9 mo and five-year DFS rate of 0%, versus a median DFS greater than follow-up period of 5 y and DFS rate of 88% for those with T2 tumors and ALN CD4 population of 7.0% or above (p=0.01) (FIG. 4C). By immune profiling of the entire study population (n=77), median DFS for the unfavorable CD4 and CD1a profiles among 33 patients with T2 tumors were both 24 mo with DFS rates of 13% and 0.0%, respectively. In contrast, favorable ALN CD4 and CD1a profiles portended DFS rate of 94% and 86%, respectively. DFS according to CD4 and CD1a immune profiles was superior to all other clinicopathologic characteristics, the most predictive characteristic being size of SLN metastasis (permuted log-rank test, ALN CD4, p<0.001; ALN CD1a, p<0.001; and size of SLN metastasis, p=0.03). Furthermore, ALN immune profiles of CD4 or CD1a cells were significantly superior to prognostic capacity by amount of local metastatic tumor burden (number of tumor-involved ALNs, p>0.05) among patients with T2 tumors.

By immune profiling, median DFS for the unfavorable CD4 and CD1a profiles among patients with T2 tumors, were both 24 months with DFS rates of 10.5% and 5.9%, respectively. Favorable ALN immune profiles portended a more favorable DFS rate of 91.7% and 88.5% for CD4 and CD1a among 45 patients with T2 tumors. DFS according to CD4 and CD1a immune profiles was superior to all other clinicopathologic characteristics (FIGS. 4A and 4C)(permuted log-rank test, P<0.001, P<0.001, and P=0.063). Further, ALN immune profiling by CD4 or CD1a are significantly superior to prognostic capacity, among patients with T2 tumors, by number of tumor-involved ALNs (P=0.192).

We also examined ALN immune profile as a prognostic tool for T1 tumors. We first determined the best current clinicopathologic predictor of disease recurrence in 41 patients with T1 tumors among our study population. This characteristic, percent of tumor involvement within the SLN, was an inferior predictor to immune profiling by ALN CD4 and CD1a (permuted log-rank test, percent tumor involvement in SLN, p=0.049; CD4, p<0.001; and CD1a, p=0.001; FIG. 4B). By ALN immune profiling among patients with T1 tumors, median DFS for the unfavorable CD4 and CD1a profiles were both 36 mo with DFS rates of 20% and 29%, respectively. Favorable ALN immune profiles portended a significantly more favorable DFS rate of 88% and 81% for CD4 and CD1a among patients with T1 tumors. Thus, for patients with T1 tumors, DFS according to CD4 and CD1a immune profiles was also superior to current clinicopathologic characteristics, including the number of tumor-involved ALNs (p. 0.05).

Relationships between Immune Profile and Metastasis in SLN and ALN. To address potential mechanisms of immune changes in breast cancer-draining lymph nodes, we further explored the dependence of immune profile changes on nodal tumor metastasis in SLNs and ALNs. Immune profile thresholds determined from ROC curve analysis of training set series 1 lymph nodes (CD4 at 7%, CD1a at 0.6%) were applied to SLNs from training set series 2 and ALNs from training set series 2 and the test set. While all of the series 1 SLNs were tumor-involved, only 50% of the series 2 SLNs were involved, making such an analysis possible for both SLN and ALN. Lymph nodes were segregated based on immune profile changes and nodal metastasis (Table 5). Among the 18 SLNs, all nine (100%) tumor-involved SLNs showed decreased percentages of CD4 cells, and 77.8% showed decreased percentages of CD1a cells. Conversely, 81.8% and 77.8% of SLNs with relatively normal percentages of CD4 cells and CD1a cells, respectively, were tumor-free. X₂ testing for CD4 and CD1a, with p-values of less than 0.001 and 0.017, respectively, demonstrate the strength of relationship between tumor involvement and immune profile in SLNs. Importantly, ALN analysis of 75 nodes from training set series 2 and the test, 24 of which were tumor-involved, did not demonstrate a similar effect of nodal tumor status on nodal immune profile (Table 5). Of the 24, 11 (46%) tumor involved ALNs exhibited preserved CD4 percentages, and 14 (58%) exhibited preserved CD1a percentages. Furthermore, of 51 tumor-free ALNs, 21 (41%) and 23 (45%) exhibited decreased percentages of CD4 or CD1a cells, respectively. Hence, among these ALNs, no statistically significant association was found between decreased CD4 or CD1a populations and nodal tumor involvement (p-values 0.298 and 0.784, respectively). To address the dependence of ALN immune profile on nodal tumor status, we directly compared the immune profiles of series 1 and series 2 ALNs from the same patient. Of 27 paired ALNs, seven were discordant (one tumor-involved and one tumor-free), allowing us to address whether nodal metastasis is the dominant cause of ALN immune profile changes within individuals. Interestingly, the variance between discordant ALN pairs from the same patients was the same or even less than the variance between concordant ALN pairs (both tumor-involved or both tumor free) (Table 6). This further supports the possibility that ALN immune profile change is driven by a separate process from nodal metastasis.

Finally, the independent predictors of DFS are shown in Table 7. The most significant independent predictors were percent of CD1a and CD4 cells in the ALN (hazards ratios of 0.42 and 0.93, respectively). Tumor size displayed a trend with recurrence (although not significant at p, 0.05), with a hazards ratio of 1.18. Neither the percent of tumor within the analyzed ALN, nor the size of tumor metastasis within the SLN, were associated with DFS by Cox proportional hazards modeling. These findings point to the intriguing possibility that immune profile changes and nodal metastasis may be independent processes in ALN. This is in contrast to SLN, in which immune profile changes appear dependent on nodal metastasis. Importantly, our data show that ALN immune profile—not SLN immune profile (see Table 3) or ALN metastasis (Table 8)—predicts DFS in breast cancer.

TABLE 5
ALN Immune Profile, Tumor Stage, and Disease-free Survival

| | Immune Profile of Patients | Immune Profile of Patients | |
|---|---|---|---|
| | Disease-free, | with Recurrent Disease, | Wilcoxon rank |
| | % of lymph node | % of lymph node | sum test |
| Cell Population | (mean ± SE) | (mean ± SE) | P-value |
| All ALNs | | | |
| ALN Immune Profile | | | |
| (n = 104)* | | | |
| CD4 % | 22.00 ± 1.60 | 6.15 ± 1.21 | <0.001 |
| CD8 % | 12.96 ± 0.83 | 7.58 ± 1.02 | <0.001 |
| CD1a % | 3.10 ± 0.28 | 0.47 ± 0.09 | <0.001 |
| Infiltrating tumor | 1.48 ± 0.64 | 1.62 ± 0.49 | 0.109 |
| cells %§ | | | |
| ALL ALNs - T1 Tumors | | | |
| ALN Immune Profile | | | |
| (n = 56)† | | | |
| CD4 % | 21.32 ± 2.14 | 7.53 ± 2.21 | <0.001 |
| CD8 % | 13.26 ± 1.16 | 9.70 ± 1.70 | 0.045 |
| CD1a % | 2.83 ± 0.35 | 0.66 ± 0.18 | <0.001 |
| Infiltrating tumor | 1.85 ± 0.99 | 1.04 ± 0.55 | 0.081 |
| cells %§ | | | |
| All ALNs - T2 Tumors | | | |
| ALN Immune Profile | | | |
| (n = 43)‡ | | | |
| CD4 % | 23.02 ± 2.41 | 4.90 ± 1.56 | <0.001 |
| CD8 % | 12.52 ± 1.14 | 5.77 ± 1.41 | <0.001 |
| CD1a % | 3.51 ± 0.46 | 0.32 ± 0.06 | <0.001 |
| Infiltrating tumor | 0.92 ± 0.57 | 1.79 ± 0.82 | 0.750 |
| cells %§ | | | |

*All ALNs, 104 axillary lymph nodes from the training set ALN series 1 (n = 29), ALN series 2 (n = 27), ALN test set (n = 48). 44 of 104 ALNs selected from patients with recurrent disease with 60 months of follow-up.
§Percent of ALN occupied by infiltrating breast tumor cells.
†All ALNs selected from patients with T1 tumors, 18 of 56 patients with recurrent disease with 60 months of follow-up.
‡All ALNs selected from patients with T2 tumors, 24 of 43 patients with recurrent disease with 60 months of follow-up.
ALN, axillary lymph node.

TABLE 6
Sentinel and Axillary Lymph Node Immune Profile and Nodal Metastases

| | Tumor-free | Lymph Tumor-involved | Lymph Correlation | X2 test§ |
|---|---|---|---|---|
| Cell Population | Node (n, %) | Node (n, %) | coefficient§ | P-value |
| Sentinel Lymph Node | | | | |
| (n = 18)† | | | | |
| CD4 % | | | −0.798 | <0.001 |
| <7%* (n = 7) | 0, 0.0 | 7, 100.0 | | |
| =7% (n = 11) | 9, 81.8 | 2, 18.2 | | |
| CD1a % | | | −0.566 | 0.017 |
| <0.6%* (n = 9) | 2, 22.2 | 7, 77.8 | | |
| =0.6% (n = 9) | 7, 77.8 | 2, 22.2 | | |
| Axillary Lymph Node | | | | |
| (n = 75)‡ | | | | |
| CD4 % | | | −0.122 | 0.298 |
| <7%* (n = 34) | 21, 61.8 | 13, 38.2 | | |
| =7% (n = 41) | 30, 73.2 | 11, 26.8 | | |
| CD1a % | | | 0.032 | 0.784 |
| <0.6%* (n = 33) | 23, 69.7 | 10, 30.3 | | |
| =0.6% (n = 42) | 28, 66.7 | 14, 33.3 | | |

§Correlation coefficient and X2 test for tumor-free lymph node immune profile CD4 and CD1a thresholds versus tumor-involved lymph node immune profile CD4 and CD1a thresholds.
†Sentinel lymph nodes - 9 tumor-involved from training set series 2 (n = 18).
‡Axillary lymph nodes - 7 tumor-involved from training set series 2 (n = 27), and 17 tumor-involved from test set (n = 48); total 24 of 75 axillary nodes are tumor-involved.
*Downregulated immune profile by CD4 % or CD1a % nodal surface area thresholds determined from training set analysis.

TABLE 7
Cox Proportional Hazards Model for DFS

| | Hazard | 95% Confidence Interval | | |
|---|---|---|---|---|
| Variable | Ratio | Lower | Upper | p-Value |
| ALN % CD1a | 0.42 | 0.239 | 0.738 | 0.003 |
| ALN % CD4 | 0.93 | 0.877 | 0.990 | 0.023 |
| Tumor size | 1.18 | 0.989 | 1.407 | 0.065 |
| ALN % tumor involvement | 0.01 | 0.001 | 3.617 | 0.581 |
| Size of SLN metastasis | 1.1 | 0.566 | 2.131 | 0.783 |

77 patients, 33 with recurrent disease during follow-up of 5 y.

TABLE 8
ALN Immune Profile, Tumor Stage, and DFS

| | | | | All ALNs, T1 Tumors | | | | All ALNs, T2 Tumors | |
|---|---|---|---|---|---|---|---|---|---|
| | All ALNs (In = 77)^{a} | | | (n = 41)^{b} | | | | (n = 33)^{c} | |
| | | Immune | | | Immune | | | Immune | |
| | Immune | Profile of | | Immune | Profile of | | Immune | Profile of | |
| | Profile of | Patients | | Profile of | Patients | | Profile of | Patients | |
| | Patients | with | | Patients | with | | Patients | with | |
| | Disease- | Recurrent | | Disease- | Recurrent | | Disease- | Recurrent | |
| | free, | Disease, | | free, % | Disease, | | free, % | Disease, | |
| | % of | % of | Wilcoxon | of | % of | Wilcoxon | of | % of | Wilcoxon |
| | Lymph | Lymph | Rank | Lymph | Lymph | rank | Lymph | Lymph | Rank |
| | Node | Node | Sum | Node | Node | Sum | Node | Node | Sum |
| Cell | (Mean ± | (Mean ± | Test p- | (Mean ± | (Mean ± | Test p- | (Mean ± | (Mean ± | Test p- |
| Population | SE) | SE) | Value | SE) | SE) | Value | SE) | SE) | Value |
| CD4 | 23.40 ± | 4.80 ± | <0.001 | 23.67 ± | 6.55 ± | <0.001 | 23.01 ± | 3.15 ± | <0.001 |
| | 1.75 | 0.96 | | 2.43 | 1.97 | | 2.52 | 0.57 | |
| CD8 | 14.20 ± | 6.42 ± | <0.001 | 14.35 ± | 8.69 ± | 0.041 | 13.97 ± | 4.40 ± | <0.001 |
| | 0.99 | 0.79 | | 1.44 | 1.41 | | 1.27 | 0.61 | |
| CD1a | 3.35 ± | 0.43 ± | <0.001 | 3.07 ± | 0.60 ± | <0.001 | 3.77 ± | 0.28 ± | <0.001 |
| | 0.33 | 0.09 | | 0.42 | 0.18 | | 0.51 | 0.08 | |
| Infiltrating | 1.90 ± | 1.83 ± | 0.226 | 2.37 ± | 1.30 ± | 0.355 | 1.21 ± | 2.27 ± | 0.556 |
| tumor cells^{d} | 0.85 | 0.58 | | 1.40 | 0.70 | | 0.75 | 1.00 | |

^{a}All individuals, 77 axillary lymph nods from the training set (n = 29). Of 77 patients, 33 were selected from patients with recurrent disease with 5 y of follow-up.
^{b}All ALNs selected from patients with T1 tumors, 15 of 41 patients with recurrent disease with 5 y of follow-up.
^{c}All ALNs selected from patients with T2 tumors, 15 of 33 patients with recurrent disease with 5 y of follow-up.
^{d}Percent of ALN occupied by infiltrating breast tumor cells.
A single ALN from each individual within the learning set was randomly selected from ALN series 1 or ALN series 2.

It is now widely accepted that the status of tumor-draining lymph nodes significantly predicts clinical outcome in breast cancer. However, current clinical practice involves only histological examination of such nodes for the presence or absence of tumor, largely ignoring the immunological nature of lymph nodes in cancer. As the systemic immune response is clearly influenced by tumor progression, immune profile changes in early sites of immune system-cancer interactions, i.e., tumor-draining nodes, may represent a sensitive indicator of tumor metastasis. More significantly, the nature of such immunological changes may provide additional biological and prognostic information.

In this study, we analyzed the lymph node immune profiles in 77 breast cancer patients with tumor-involved SLNs, 42 of which had tumorpositive ALNDs. Importantly, in 5 y of follow-up, 33 patients had disease recurrence, allowing us to correlate nodal immune profile with clinical outcome. Four patients had SLNs containing isolated tumor cells (0.2 mm or smaller) detected by only IHC—these patients developed disease recurrence, supporting the clinical significance of IHC-only positive SLNs. As in other studies, mesenteric nodes from patients with benign disease were used as comparisons, since axillary nodes are rarely excised for nonmalignant conditions; immune profile of control nodes paralleled literature standards.

Importantly, new computer based imaging techniques provided high-resolution image acquisition of the entire nodal surface. We acquired a total of 160-4,130 images (2003 magnification) per nodal section, while prior studies based their results on only 5-20 images per section. By such detailed, automated analysis of SLNs and ALNs, we identified unique patterns in the degree of CD4 helper T cell, CD8 cytotoxic T cell, and CD1a dendritic cell decreases relative to each other and controls. Even tumor-free ALNs exhibited changes in immune profile, with suppression of CD4 and CD8 T cells relative to controls. In contrast, tumor-free ALNs exhibited higher dendritic cell populations than controls, and this elevation was more prominent in tumor-free ALNs from patients with positive ALNDs than from patients with negative ALNDs. This demonstrates that perturbations of the immune profile in tumor-free ALNs are dynamic and may occur before gross nodal metastasis.

Our findings extend prior studies in melanoma, lung, head and neck, gastric, and breast cancer, which linked immune downregulation only to tumor invasion, and also show that the relationship between increasing tumor invasion and changes in immune profile is not a simple linear one. While prognostic factors, including lymph node metastasis, tumor size, and histological grade, for breast cancer recurrence and overall survival are well established, few studies have thoroughly examined the influence of immune profile on clinical outcome.

The present data provide the first demonstration of the clinical significance of T helper and dendritic profiles within tumor draining nodes of breast cancer patients in predicting DFS. The ALN immune profile appears much less influenced by the presence of intranodal metastatic tumor cells. As the direct (tumor infiltration) and indirect (altered cytokine profile) effects of cancer progression alter the nodal environment, the predictive capacity of the SLN immune profile may become diminished, and the influence of infiltrating tumor is augmented. This is analogous to observations in melanoma, in which proximity to primary tumor is the dominant determinant of immune profile.

By profiling ALNs, we observed a predictive accuracy of recurrence by dendritic and T cell populations that is superior even to the predictive accuracy of tumor involvement within the identical node. Furthermore, ALN immune profile predicted recurrence independent of presence or absence of metastasis on ALND. Therefore, a single axillary (nonsentinel) node, selected regardless of tumor involvement within the node or the overall status of all other nodes from the patient's ALND, contains a unique immune profile of potential prognostic value.

In summary, our findings demonstrate that changes in the immune profile of breast cancer-draining lymph nodes accompany, and may precede, tumor invasion. Perturbation of the SLN immune profile, while highly correlated with the presence of infiltrating metastases, does not add further predictive value in patient prognosis. In contrast, our data show that ALN immune profile does predict DFS much better than it does ALN nodal metastasis. The prognostic value of ALNs is highlighted by the capacity of immune profiling of a single, randomly selected ALN to stratify risk of recurrence among early stage breast cancer. Immune profiling of ALN CD4 T cells and CD1a dendritic cells among T1 and T2 tumors dramatically differentiates a population at high risk of recurrence significantly better than all available clinicopathologic patient characteristics. The additional prognostic significance of the immune profile among this subset of breast tumors is not possible by other patient, tumor, or lymph node characteristics.

These findings support that a subset of patients may be at higher risk of recurrence due to the extent of immune profile changes, and may therefore justify consideration of more aggressive therapy. Finally, our findings offer mechanisms underlying breast cancer's poor immunogenicity, due to either deficient co-stimulation secondary to low helper T cell populations, or inability to activate T cells as a result of down-regulation of antigen-presenting dendritic cells. Strategies to augment T cell and dendritic cell populations and function within tumor-draining nodes may increase the potential for an effective immune response and thus improve clinical outcome among breast cancer patients.

### EXAMPLE 2

### Dendritic and T Cell Dysregulation in Axillary Lymph Nodes Predict Breast Cancer Recurrence

The capacity of tumor cells to evade the host immune response is now well established. However, the specific in vivo tumor-immune interactions leading to immune tolerance induction remain poorly understood. Within the primary tumor, detailed ex vivo analyses by immunohistochemistry of ovarian, gastrointestinal, bladder, lung, and breast cancers have demonstrated a significant lymphocytic infiltrate, many of which express an immune suppressing phenotype characterized as CD4 T regulatory cells. Further primary tumor analysis by cell phenotyping in combination with architectural modeling has demonstrated an abnormally increased ratio of immature to mature dendritic cells preferentially located along the primary tumor margin. Though the immunophenotype within the primary tumor supports immune tolerance, it is unclear if this is a result of, or results in, tumor-immune modulation at other systemic sites such as peripheral blood, bone marrow, and lymph nodes.

Whether tumor-draining lymph nodes are invaded by tumor cells is a key determinant of clinical outcome in breast cancer. As lymph nodes are immunologically active tissues, they represent important sites of tumor-immune interactions and initiation of the anti-tumor immune response. As shown in Example 1, T cell and dendritic cell populations within axillary lymph nodes of patients with breast cancer are significantly decreased in patients who relapsed, suggesting that immune dysregulation observed at the primary tumor also occurs within locoregional nodes. That perturbations of T cell populations occur in tumor-free nodes from patients with malignancy suggests tumor-dependent immune modulation occurs independent of cell-cell contact. One proposed mechanism of decreased T lymphocyte populations within tumor-draining nodes compared to nodes from healthy subjects is an increased population of regulatory T cells which inhibit lymphocyte proliferation.

No study thus far has examined the architectural variability in lymphocyte populations and phenotypes as related to lymph node-infiltrating tumor cells and clinical outcome. The location of tumor-dependent immune modulation has significant sequelae, given the critical role of lymph nodes in activation of the immune response. As altered immune populations originally observed in the primary tumor have subsequently been observed in tumor-draining nodes, we hypothesize that tumor-draining nodes may contain similar architectural and phenotypic perturbations of the immune response. Of clinical importance, immune modulation within the primary tumor and tumor-draining nodes has been shown to predict patient prognosis, specifically disease-free survival. If architectural and phenotypic changes in immune cells are present within tumor-draining nodes, they may provide both prognostic value and further insight into the biological alterations of tumor-dependent immune modulation.

Methods

STUDY PATIENTS. 29 breast cancer patients aged 29-76 years treated at Stanford University Medical Center between February 1997 and January 1999 were evaluated. Patients who underwent ALN dissection due to tumor-involved SLN biopsies, as is standard clinical practice, with five-year clinical outcome data available were selected. Two ALNs from each patient, as defined by the operative and pathology reports were selected by an independent member of the Stanford Pathology Department blinded to study design and ALN metastasis status. No selection criteria, such as nodal size or appearance, were used in ALN selection in order to avoid a biased ALN sample set. To provide an average immune profile, ten control nodes, a single mesenteric node per control subject, were similarly examined from patients with benign disease without a history of malignancy or immune disorder. All samples were collected from Stanford Department of Pathology Specimen Bank as coded specimens under a protocol approved by the Stanford University Medical Center Institutional Review Board.

All subjects were untreated and without a history of cancer or immune disorder prior to breast cancer diagnosis and SLN biopsy. Following surgical management, patients received adjuvant therapy as determined by their medical and radiation oncologists. The duration of disease-free survival was the time between initial diagnosis and first recurrence. All patients received SLN and ALN removal in conjunction with removal of primary tumor within 44 days of initial diagnosis. Initial diagnosis was performed by needle aspiration or core biopsy in the majority of cases. Final diagnosis was confirmed from the pathologic evaluation of the primary tumor from the lumpectomy specimen. The average difference between time of diagnosis and surgery was 12.3 days. We chose to use time of diagnosis rather than time of surgery to determine clinical outcome as we are measuring the interaction between tumor and immune composition of local nodes versus the influence of surgery on outcome. All recurrences were based on documentation of local or systemic disease during a follow-up period of five years after which data were censored. We recorded and verified patient, tumor, and lymph node characteristics.

IMMUNOSTAINING. Serial, adjacent, tissue sections, 3 μm thick, were cut from formalin-fixed, paraffin-embedded nodes. HES and IHC were performed after antigen retrieval using Biogenex Genomx i1000 (San Ramon, Calif.). Antibodies used included anti-CD4 (1/20, Novacastro), anti-CD8 (1/25, Dako), anti-CD25 (1/50, Novus Biologicals), anti-CD1a (1/100, Dako), anti-CD83 (1/50, Serotec), antigranzyme B (1/20, GrB-7, Chemicon), anti-FOXP3 (1/100, Abcam), anti-AE1/AE3 (1/25, Biogenex), and as secondary antibody—EnVision dextran kit (1/5, Dako). Optimal concentrations were determined, and tested in sample node sections. Double staining using 3′3′ diaminobenzidene, VIP (Vector Laboratories), and a light counterstain with Mayer's hematoxylin (Innogenex) was performed for lymphocyte population of interest with and colocalization of tumor cells. Isotype-matched antibodies were used as negative controls. All slides for the respective antibody were stained in the same run. Presence of metastasis was verified by HES and IHC on four sections per node by two blinded investigators trained in breast cancer pathology. Area of node occupied by each immune cell type and by tumor was determined through computerized image acquisition and analysis software, BLISS (Bacus Lab Inc., Slide Scanner).

Prior image analyses determine cell count and area from an average of five to 20 high-power fields. Using BLISS we acquired 160-833 sequential images at 100× to 200× of the entire lymph node section, which were sequenced together by Metamorph Imaging System (Universal Imaging). Objectives were calibrated to transform image pixels to μm. Control nodes were examined to standardize thresholds of each stain for cell of interest. Using an automated Metamorph script, standardized thresholds were applied with Metamorph log set to record areas occupied by cell of interest, tumor, and of entire node for all samples, thus minimizing any potential operator bias.

For architectural pattern analysis, four subdivisions of each tumor-involved lymph node were analyzed as mapped in FIG. 2; the entire nodal surface area, the tumor margin, the tumor border, and within the infiltrating tumor focus. Each nodal area was partitioned and analyzed for cell type and population as follows: I. entire nodal surface area—all stained areas of the node; II. tumor margin—a rim of one medium-powered field (100×) around the tumor focus; III. tumor border—a series of contiguous high-powered fields (200×) with the center of each image at the interface of infiltrating tumor and nodal lymphocytes, circling the tumor focus; and, IV. tumor focus×all areas within the tumor border. Lymphocyte populations are expressed as percent lymph node surface area with a correction for the area occupied by tumor cells. This was performed in order to minimize variability in lymphocyte populations based on variance in nonlymphocyte populations, such as infiltrating tumor cells.

STATISTICAL ANALYSIS. Univariate and multivariate analyses including logistic regression tested predictive capacity of patient characteristics. Cell populations within each of four lymph node regions and stratified by nodal status and disease recurrence were compared by analysis of variance with correction for multiple comparisons by Tukey's honestly significantly different test. Population comparisons within lymph node regions were calculated by Wilcoxon rank sum test. Two-sided p<0.05 corrected for multiple comparisons was considered a statistically significant difference. Relationship between FOXP3:CD4 and CD83:CD1a was determined by Spearman's rho correlation test. For analyses we used R statistical package.

Results

Patient and Lymph Node Characteristics. Characteristics of the study population are shown in Table 9. Eleven of 29 patients developed recurrent disease within 60 months. Two of 11 recurrences, 18%, occurred at a distant site, and 10 of 11 developed locoregional relapse, 91%, with one patient at time of relapse found to have both local and distant disease. Of 29 ALNs selected (one per patient), 16 were tumor-involved, 55.2%, allowing architectural pattern analysis of intranodal, tumor-lymphocyte interactions. All 29 initially selected ALNs and an additional ALN from each patient were analyzed for FOXP3 expression and dendritic cell maturation. Twenty-two of the additional 29 ALNs, 75.9%, were tumor-involved.

| | Patients, | |
|---|---|---|
| | number | |
| Characteristic | (n = 29) | % |
| Patient Characteristics | | |
| Age (years) | 52 (29-76) | |
| Age < 51 | 16 | 55.2 |
| Age ≧ 51 | 13 | 44.8 |
| Stage | | |
| Stage IIA | 11 | 37.9 |
| Stage IIB | 11 | 37.9 |
| Stage IIIA | 7 | 24.1 |
| Primary Tumor Characteristics | | |
| Tumor size (mm) | 21.4 (2.0-75) | |
| Histological tumor stage | | |
| T1a | 1 | 3.4 |
| T1b | 4 | 13.8 |
| T1c | 11 | 37.9 |
| T2 | 11 | 37.9 |
| T3 | 2 | 6.9 |
| Tumor grade | | |
| G1: Low combined histologic grade | 2 | 6.9 |
| G2: Intermediate combined histologic grade | 17 | 58.6 |
| G3: High combined histologic grade | 10 | 34.5 |
| ER status | | |
| Negative | 12 | 41.4 |
| Positive | 17 | 58.6 |
| PR status | | |
| Negative | 13 | 44.8 |
| Positive | 16 | 55.2 |
| HER2/neu expression | | |
| Not overexpressed | 18 | 62.1 |
| Equivocal | 3 | 10.3 |
| Overexpressed | 8 | 27.6 |
| Angiolymphatic invasion | | |
| None | 19 | 65.5 |
| Present | 10 | 34.5 |
| Sentinel Lymph Node Characteristics | | |
| Number of tumor-free sentinel lymph nodes | 0.86 (0-4) | |
| 0 | 16 | 55.2 |
| ≧1 | 13 | 44.8 |
| Size of SLN metastases | | |
| Isolated tumor cells ≦ 0.2 mm | 5 | 17.2 |
| Micrometastases ≦ 2 mm | 11 | 37.9 |
| Macrometastases > 2 mm | 13 | 44.8 |
| Extracapsular extension | | |
| None | 26 | 89.7 |
| Present | 3 | 10.3 |
| Sentinel lymph node metastases identification | | |
| Hematoxylin and eosin staining | 19 | 65.5 |
| Immunohistochemistry | 10 | 34.5 |
| Nodal stage | | |
| N1 | 23 | 79.3 |
| N2 | 6 | 20.7 |
| Non-sentinel Lymph Node Characteristics | | |
| Number of tumor-free non-sentinel lymph | 6.72 (1-19) | |
| nodes | | |
| <7 | 16 | 55.2 |
| ≧7 | 13 | 44.8 |
| Number of tumor-involved nonsentinel lymph | 1.72 (0-11) | |
| nodes | | |
| 0 | 13 | 44.8 |
| ≧1 | 16 | 56.2 |
| Clinical Outcome | | |
| Recurrent disease^{†} | | |
| Yes | 11 | 37.9 |
| No | 18 | 62.1 |

^{†}Recurrence during follow-up of 60 months.

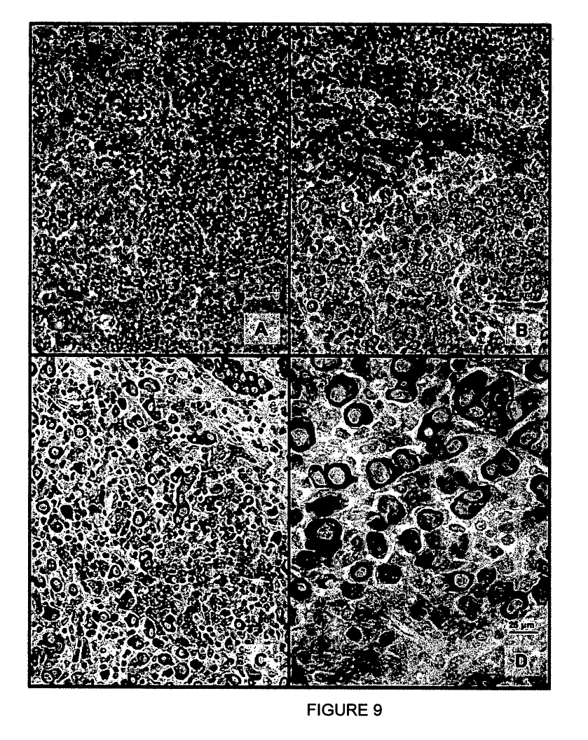

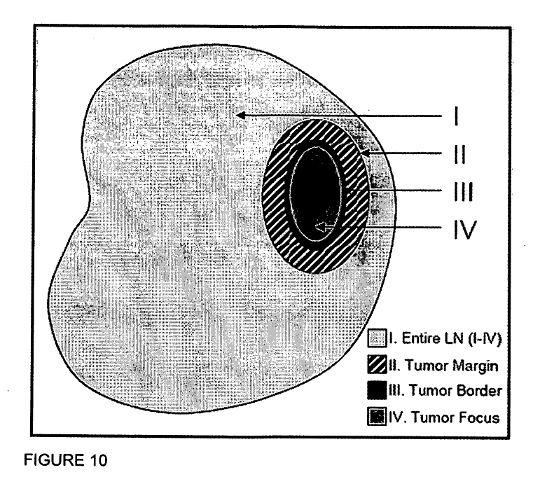

CD4 and CD8 T Cell Architectural Pattern Analysis. Observational analysis of location of CD4 and CD8 T cells within 16 tumor-involved nodes illustrated patterns of T cell location relative to lymph node-infiltrating breast cancer cells. Populations of CD4 T cells appeared similarly distributed throughout lymph node sections (FIG. 9A). CD8 T cells, however, appeared in higher density near and within the tumor focus of infiltrating breast cancer than peripherally in uninvolved portions of the node (FIG. 9B, tumor border; FIGS. 9C and 1D, tumor focus). Specific subdivisions of cell locations within the node relative to the focus of infiltrating tumor cells were defined in order to quantify the variation in T cell populations (FIG. 10).

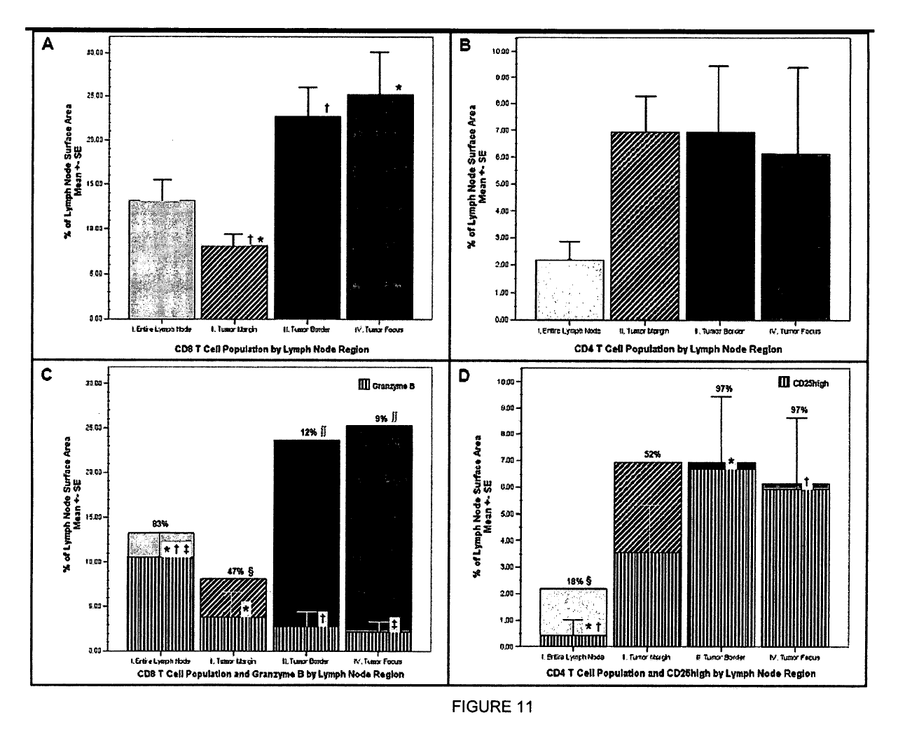

Four regions, as detailed in the methods, including the entire lymph node, tumor margin, tumor border, and tumor focus, allowed rapid assessment of architectural variance in cell distribution. CD8 T cells occupied 13.19% of the entire nodal surface area, and increased in relative density from the tumor margin to within the tumor focus; 8.12% at the tumor margin*†, 23.61% at the tumor border†, and 25.26% within the tumor focus* (FIG. 11A)(†p=0.011, *p=0.004). CD4 T cells occupied 2.18% of the entire nodal surface area, without a significant change in relative density compared to tumor margin, 6.95%, tumor border, 6.95%, or within the tumor focus, 6.13% (FIG. 11B)(p>0.05 for all comparisons).

Granzyme B and CD25 Architectural Pattern Analysis. Adjacent sections from 16 tumor-involved ALNs analyzed initially for pattern of CD4 and CD8 T cell interactions with infiltrating tumor cells were stained for CD25 and granzyme B, respectively. Both percent of lymph node surface area and percent of CD4 or CD8 T cells expressing CD25 or granzyme B varied based on location relative to focus of infiltrating tumor cells. Granzyme B expression decreased with increasing proximity to and within the tumor focus; 10.97% in the entire lymph node*†‡, 3.81% at the tumor margin*, 2.93% at the tumor border†, and 2.27% within the tumor focus‡ (FIG. 11C)(*p=0.001, †p=0.001, ‡p=0.001). This represents a statistically significant decrease in percent of CD8 T cells with granzyme B expression compared to the overall CD8 population by lymph node region: 83% in the entire lymph node, 47% at the tumor margin, 12% at the tumor border, and 9% within the tumor focus (p=0.323, p=0.032, p<0.001, p<0.001, respectively). Tumor-involved lymph node sections illustrated a bimodal pattern of CD25 staining. A subset of lymphocytes stained intensely with CD25, referred to as CD25^{high}. The percent of lymph node surface area staining CD25^{high} increased from entire lymph node*†, 0.391%, compared to percent at the tumor margin, 3.62%, at the tumor border*, 6.74%, or within the tumor focus†, 5.95% (FIG. 11D)(*p=0.030, †p=0.071). Represented as the percent of CD4 T cells expressing CD25^{high}, a similar increase was observed by lymph node region; 18% in the entire lymph node, 52% at the tumor margin, 97% at the tumor border, and 97% within the tumor focus (p=0.035, p=0.073, p=0.862, p=0.381, respectively).

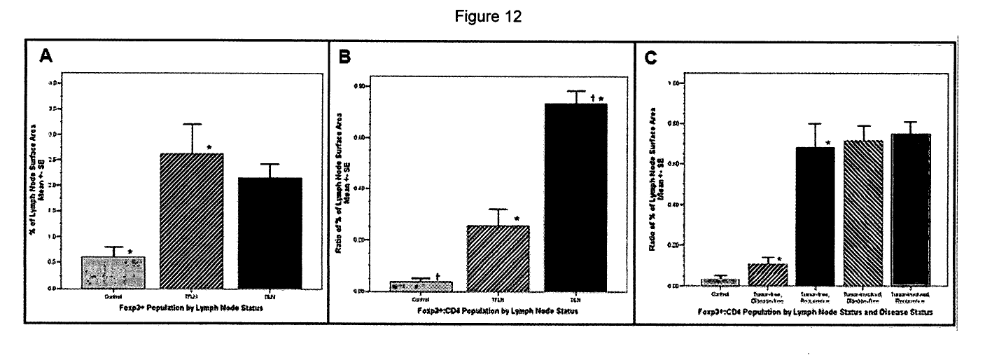

Nodal FOXP3 Expression and Disease-free Survival. Two ALNs per patient were analyzed for tumor-involvement status determined by epithelial cell stain AE-1/AE-3 and for expression of transcription factor FOXP3. Percent of lymph node surface area expressing FOXP3 was significantly increased in nodes of patients with breast cancer, n=58, versus nodes of controls, n=10, regardless of tumor invasion. Surprisingly, the largest FOXP3+ population was found in tumor-free nodes of patients with breast cancer (2.64%) compared to tumor-involved nodes (2.16%, p>0.05) and control nodes (0.61%, p=0.027)(FIG. 12A). However, the ratio of FOXP3+ to CD4+ staining, or relative FOXP3+ population, was greatest in tumor-involved nodes (0.74 or 74%) compared to tumor-free nodes (0.26, p<0.001) and controls (0.04, p<0.001)(FIG. 12B). Relative FOXP3+ population and nodal metastasis status was analyzed with stratification based on disease-free survival (FIG. 12C). Relative FOXP3+ populations were high in tumor-involved lymph nodes from patients with or without disease recurrence, 0.76 and 0.72, n=17 and 21 respectively. Importantly, relative FOXP3+ populations within tumor-free nodes were also high from patients with recurrence, 0.68, n=5. In contrast, controls, n=10, and tumor-free nodes from patients without disease recurrence, n=15, contained low relative FOXP3+ populations (0.04 and 0.11, respectively). This represents a significant difference between relative FOXP3+ population in tumor-free nodes from patients with versus without disease recurrence (0.68 and 0.11, p=0.002).

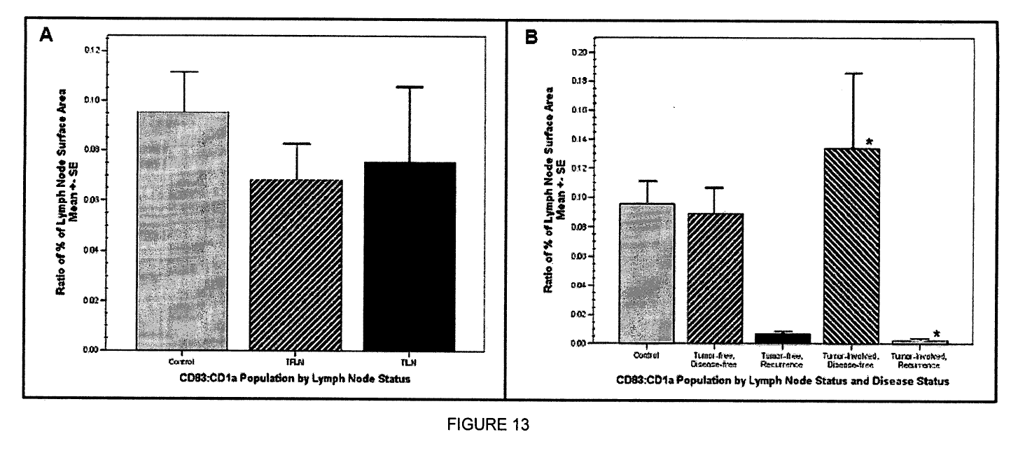

Nodal Dendritic Cell Maturation and Disease-free Survival. Two ALNs per patient with analysis for tumor-involvement by AE-1/AE-3 staining were analyzed for dendritic cell maturation status by CD1a and CD83 expression. As CD83 is expressed by mature dendritic cells and CD1a is expressed by both immature and mature dendritic cells, the ratio of CD83:CD1a expression provided a quantitative value of dendritic cell maturation status, with higher values suggesting a greater percent of nodal dendritic cells are of a mature phenotype. A more mature dendritic cell profile was present in all control nodes (0.096) compared to tumor-free or tumor-involved nodes of patients with breast cancer (0.069 and 0.075, respectively)(FIG. 13A). Analysis based on disease-free survival identified a more mature dendritic cell profile among lymph nodes from patients without recurrent disease, regardless of status as tumor-free or tumor-involved (0.089 and 0.134, respectively)(FIG. 13B). Lymph nodes from patients with recurrent disease, again regardless of tumor involvement status, displayed a more immature dendritic cell profile (0.007 and 0.003 for tumor-free and tumor-involved nodes of patients with recurrent disease). Tumor-involved nodes from patients with disease recurrence contained a more immature dendritic cell profile relative to nodes with an identical metastatic status that were selected from patients disease-free at five years (0.003 and 0.134, p=0.036).

Nodal Dendritic Cell Maturation and FOXP3 Expression. The relationship between FOXP3 expression within tumor-draining nodes as well as controls and maturation status of nodal dendritic cells was examined by correlation comparison of relative FOXP3+ population (ratio of FOXP3+ population to CD4+ population) and dendritic cell maturation (ratio of CD83+ population to CD1a+ population). Degree of dendritic cell maturation correlated with relative FOXP3+ population (r2=−0.28, p=0.011). Though the correlation is relatively weak, its statistical significance suggests an inverse relationship between nodal dendritic cell maturation and relative FOXP3 expression: lymph nodes with a mature dendritic cell profile contain less relative FOXP3 expression.

Perturbations of T cell and dendritic cell populations within axillary nodes draining primary breast tumors suggest immune response inhibition is a critical component of tumor growth and metastasis. By investigating the detailed architectural and phenotypic appearance of immune cells within tumor-draining lymph nodes, we observed previously unreported immunologic abnormalities which provide insight into the process of in vivo immune response inhibition in breast cancer. An initial CD8-mediated anti-tumor immune response is suggested by the observation of increasing density of CD8 T cells with increasing proximity to the focus of infiltrating tumor cells. However, phenotypic analysis for potentially cytotoxic CD8 T cells revealed the inverse relationship with the lowest relative density of granzyme B expression within the tumor focus. While CD4 helper T cells appeared more uniformly distributed across the lymph node, they express high levels of CD25, suggestive of a regulatory T cell phenotype, with increasing density within the tumor focus.

Staining for FoxP3, recognized as a marker for T regulatory cells, further supports the notion of increased regulatory T cells in areas of metastatic tumor. Such cells represent a higher proportion of the total CD4 T cell population within tumor-involved than tumor-free nodes; moreover, they also represent a larger relative population among tumor-free nodes from patients with breast cancer relapse compared to those who remained disease-free. A factor contributing to the observed increase in the T regulatory cell population was an inverse correlation between relative population of T regulatory cells and maturation of dendritic cells (by CD83:CD1a ratios) within axillary nodes. Both relative FoxP3 expression and dendritic cell maturation within tumor draining lymph nodes correlate with disease-free survival, emphasizing the clinical significance of immune-inhibitory lymphocytes and impaired antigen presentation in determining patient prognosis.

These detailed architectural and phenotypic observations from direct ex vivo lymph node tissue provide insights into the biological alterations underlying tumor-induced immune modulation and ultimately, clinically significant, immune tolerance. The present findings demonstrate a relationship between size of immature dendritic cell population and proportion of CD4+ lymphocytes expressing CD25^{high}, with additional supporting evidence by FoxP3 staining within axillary nodes of patients with breast cancer. FoxP3+ T regulatory cells inhibit the proliferation and cytotoxicity of CD8 T cells, and also impair antigen presentation by dendritic cells. In murine models, depletion of the T regulatory population actually augments the sensitization of tumor-specific T cells in tumor draining nodes.

We demonstrate a direct relationship between the cellular location of tumor relative to the CD4+ regulatory T cells and granzyme B+ CD8 T cells suggesting that a cell contact-dependent mechanism is responsible. Further we observed a clinical outcome benefit, specifically disease-free survival, among breast cancer patients with decreased populations of T regulatory cells. Taken together, our results demonstrate the capacity of breast cancer to induce immune tolerance by inhibiting the nodal anti-tumor immune response. The perturbations of T lymphocyte and dendritic cell populations, architecturally as related to the location of metastatic tumor cells, and phenotypically through characterization of CD25, FoxP3, granzyme B, and dendritic cell maturation, provide clinically-based support for the progression of tumor dependent immune modulation. Importantly, these results illustrate the capacity of lymph node T regulatory cell population size and dendritic cell maturation to predict risk of breast cancer recurrence, suggesting that immune dysregulation observed at the local lymph node level is of systemic significance.

It is evident that subject invention provides a convenient and effective way of determining whether a patient will be responsive to therapy. The subject methods will provide a number of benefits, including avoidance of delays in alternative treatments, elimination of exposure to adverse effects of therapeutic antibodies and reduction of unnecessary expense. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of phenotyping a cancer, the method comprising:
combining a regional lymph node sample from a patient suspected of having an invasive cancer with specific binding members that are sufficient to quantitate dendritic cells and T cells present in said regional lymph node;
quantitating the T cells and dendritic cells to generate a test immune profile,
comparing said immune profile to a reference immune profile;
wherein said test immune profile is indicative of the phenotype of said cancer.

2. The method according to claim 1, wherein said regional lymph node is a non-sentinel lymph node.

3. The method according to claim 1, wherein said T cells are helper T cells.

4. The method according to claim 1, wherein said specific binding members are antibodies.

5. The method according to claim 4, wherein said antibodies are specific for CD4 and for CD1a.

6. The method according to claim 4, wherein said antibodies are specific for CD8.

7. The method according to claim 4, wherein said antibodies are specific for FoxP3.

8. The method according to claim 4, wherein said antibodies are specific for CD25.

9. The method of claim 4, wherein said quantitation is performed by immunohistochemistry.

10. The method of claim 4, wherein said quantitation is performed by flow cytometry.

11. The method of claim 4, wherein said quantitation is performed by ELISPOT.

12. The method according to claim 1, wherein said cancer is breast cancer and said regional lymph nodes are axillary lymph nodes.

13. The method according to claim 12, wherein the increased presence of dendritic cells and decreased presence of T cells relative to a control lymph node is indicative of a positive prognosis.

14. The method of claim 1, further comprising the step of utilizing said cancer phenotype to guide patient care.

15. A kit for use in any of the methods set forth in claim 1.
